SEQUENCE LISTING (9 sequences)

SEQ ID NO: 1 - DNA, 399 bp, Artificial Sequence
  SYNTHESIZED
atggagaagaagagcctcgccggcctgtgcttcctgttcctcgtgctcttcgtggcccaggagatcgtggtgaccgaggccctcggcatcaactgccgcggctccagccagtgcggcctctccggcggcaacctgatggtgaggatcagggaccaggcctgcggcaaccagggccagacctggtgcccaggcgagaggagggccaaggtgtgcggcaccggcaacagcatcagcgcctacgtgcagagcaccaacaactgcatcagcggcaccgaggcctgccgccacctcaccaacctcgtgaaccacggctgccgcgtgtgcggcagcgacccgctgtacgccggcaacgacgtgtccaggggccagctcaccgtgaactacgtgaacagctgctga

SEQ ID NO: 2 - protein, 132 aa, Artificial Sequence
  SYNTHESIZED
MEKKSLAGLCFLFLVLFVAQEIVVTEALGINCRGSSQCGLSGGNLMVRIRDQACGNQGQTWCPGERRAKVCGTGNSISAYVQSTNNCISGTEACRHLTNLVNHGCRVCGSDPLYAGNDVSRGQLTVNYVNSC

SEQ ID NO: 3 - DNA, 315 bp, Artificial Sequence
  SYNTHESIZED
ctcggcatcaactgccgcggctccagccagtgcggcctctccggcggcaacctgatggtgaggatcagggaccaggcctgcggcaaccagggccagacctggtgcccaggcgagaggagggccaaggtgtgcggcaccggcaacagcatcagcgcctacgtgcagagcaccaacaactgcatcagcggcaccgaggcctgccgccacctcaccaacctcgtgaaccacggctgccgcgtgtgcggcagcgacccgctgtacgccggcaacgacgtgtccaggggccagctcaccgtgaactacgtgaacagctgc

SEQ ID NO: 4 - protein, 105 aa, Ustilago maydis virus P4
LGINCRGSSQCGLSGGNLMVRIRDQACGNQGQTWCPGERRAKVCGTGNSISAYVQSTNNCISGTEACRHLTNLVNHGCRVCGSDPLYAGNDVSRGQLTVNYVNSC

SEQ ID NO: 5 - DNA, 81 bp, Artificial Sequence
  SYNTHESIZED
atggagaagaagagcctcgccggcctgtgcttcctgttcctcgtgctcttcgtggcccaggagatcgtggtgaccgaggcc

SEQ ID NO: 6 - protein, 27 aa, Artificial Sequence
  SYNTHESIZED
MEKKSLAGLCFLFLVLFVAQEIVVTEA

SEQ ID NO: 7 - protein, 127 aa, Ustilago maydis virus P4
MQIINVVYSFLFAAAMLPVVHSLGINCRGSSQCGLSGGNLMVRIRDQACGNQGQTWCPGERRAKVCGTGNSISAYVQSTNNCISGTEACRHLTNLVNHGCRVCGSDPLYAGNDVSRGQLTVNYVNSC

SEQ ID NO: 8 - DNA, 477 bp, Ustilago maydis virus P4
atgcagattataaatgtggtatactccttcctgtttgcggcagcaatgcttccagtggttcactccttagggattaattgtaggggcagctcgcaatgtgggttatccggcgggaaccttatggtccgaataagagatcaggcatgtggtaaccaaggccaaacatggtgtcctggcgaacggcgtgctaaggtctgtgggactggcaatagcatctctgcttatgttcagtctaccaacaattgtatatcagggacagaggcctgtcgccatttgactaacctcgttaaccatggttgtagagtctgtggcagcgacccactctatgccgggaatgatgtgtcccgagggcagttgactgtcaactacgtaaactcgtgttgatacggaccactatcgaagtgtgtggtatatggtagtaggacttgaggggttatagacgccaacggtggggacactgtaggggaaactggtgta

SEQ ID NO: 9 - DNA, 411 bp, Ustilago maydis virus P4
ttagggattaattgtaggggcagctcgcaatgtgggttatccggcgggaaccttatggtccgaataagagatcaggcatgtggtaaccaaggccaaacatggtgtcctggcgaacggcgtgctaaggtctgtgggactggcaatagcatctctgcttatgttcagtctaccaacaattgtatatcagggacagaggcctgtcgccatttgactaacctcgttaaccatggttgtagagtctgtggcagcgacccactctatgccgggaatgatgtgtcccgagggcagttgactgtcaactacgtaaactcgtgttgatacggaccactatcgaagtgtgtggtatatggtagtaggacttgaggggttatagacgccaacggtggggacactgtaggggaaactggtgta

(19) United States
(12) Patent Application Publication
(10) Pub. No.: US 2013/0117880 A1
(43) Pub. Date: May 9, 2013

(54) TRANSGENIC PLANTS EXPRESSING A VIRAL ANTIFUNGAL PROTEIN

(71) Applicants: Thomas James Smith, St. Louis, MO (US); Dilip Shah, St. Louis, MO (US)

(72) Inventors: Thomas James Smith, St. Louis, MO (US); Dilip Shah, St. Louis, MO (US)

(73) Assignee: DONALD DANFORTH PLANT SCIENCE CENTER, St. Louis, MO (US)

(21) Appl. No.: 13/809,997

(22) PCT Filed: Jul. 20, 2011
(86) PCT No.: PCT/US11/44601

§ 371 Date: Jan. 14, 2013

Related U.S. Application Data
(60) Provisional application No. 61/365,984, filed on Jul. 20, 2010.

(51) Int. Cl. C12N 15/82

(52) U.S. Cl. CPC C12N 15/8282
USPC 800/265; 800/301; 435/418; 800/279; 536/102; 554/1; 426/615; 426/635; 426/622

(57) ABSTRACT

Transgenic plants expressing the KP4 antifungal protein are provided which exhibit high levels of antifungal resistance. Such transgenic plants contain a recombinant DNA construct comprising a heterologous signal peptide sequence that is oper ably linked to a non-native nucleic acid sequence encoding a mature KP4 antifungal protein.

### CROSS REFERENCE TO RELATED APPLICATIONS

This application claims the benefit of priority of United States provisional application No. 61/365,984, filed Jul. 20, 2010, the disclosure of which is incorporated by reference as if written herein in its entirety.

### BACKGROUND OF THE INVENTION

1. Field of the Invention

This invention relates to the development of transgenic plants expressing recombinant proteins which are capable of inhibiting the growth and /or germination of pathogenic fungi.

Protection of agriculturally important crops from pathogenic fungi is crucial in improving crop yields. Fungal infections are a particular problem in damp climates and may become a major concern during crop storage, where such infections can result in spoilage and contamination of food or feed products with fungal toxins. Unfortunately, modern growing methods, harvesting and storage systems can promote plant pathogen infections.

Control of plant pathogens is further complicated by the need to simultaneously control multiple fungi of distinct genera. For example, fungi such as Alternaria; Ascochyta; Botrytis; Cercospora; Colletotrichum; Diplodia; Erysiphe; Fusarium; Gaeumanomyces; Helminthosporium; Macrophomina; Nectria; Peronospora; Phakopsora; Phoma; Phymatotrichum; Phytophthora; Plasmopara; Podosphaera; Puccinia; Pythium; Pyrenophora; Pyricularia; Rhizoctonia; Scerotium; Sclerotinia; Septoria; Thielaviopsis; Uncinula; Venturia; and Verticillium species are all recognized plant pathogens.

Consequently, resistant crop plant varieties or fungicides that control only a limited subset of fungal pathogens may fail to deliver adequate protection under conditions where multiple pathogens are present. It is further anticipated that plant pathogenic fungi may become resistant to existing fungicides and crop varieties, necessitating the introduction of fungal control agents with distinct modes of action to combat the resistant fungi.

The maize smut fungus, Ustilago maydis, is a global pathogen responsible for extensive agricultural losses. Given that maize is the most economically important crop in the USA—generating $48.7 billion in 2009, with approximately 35 million hectares planted—even relatively small losses can result in significant monetary impact. Moreover since maize plays a major role in current biofuel production, the importance of maize in US agriculture is only expected to increase.

Control of this infection using traditional breeding has met with limited success because natural resistance to U. maydis is organ-specific and involves numerous maize genes (Baumgarten, 2007).

Accordingly, there remains the need for improved methods for the control of Ustilago maydis, as well as other fungi, and the production of transgenic plants expressing the antifungal protein (killer protein) KP4 provides a novel approach for controlling such pathogens.

KP4 is a single polypeptide of 105 amino acids produced by the UMV4 virus that infects the P4 strain of U. maydis (Park, 1994). It is the only U. maydis toxin not processed by Kex2p, and there is no sequence similarity to other toxins (Ganesa, 1991).

Additionally there is growing evidence that KP4, and related proteins, are safe for human and animal consumption. In fact KP4 is quickly degraded within less than 60 seconds in artificial stomach fluid and its amino acid sequence is not similar to any known allergens (Schlaich, 2006). Furthermore purified KP4 had no effect on viability or on subcellular structures of human, plant, insect and hamster cell lines, or on bacterial and fungal soil communities, wheat-infesting insects, such as aphids, and “standard” soil arthropod Folsomia candida (Widmer, 2007). Finally, it is highly likely that these antifungal proteins are already in the food supply since corn smut is commonly consumed in Mexican cuisine (huitlacoche) and recent studies demonstrated that all of the samples of U. maydis taken from fields in Mexico are infected by this Totivirus family (Voth, 2006).

### SUMMARY OF THE INVENTION

This invention provides for transgenic plants capable of inhibiting the growth and development of pathogenic fungi. In some embodiments, transgenic plants can be produced by a process comprising the steps of introducing a DNA construct of the invention into a plant, a plant cell, or a plant tissue, and obtaining a transgenic plant comprising the DNA construct that expresses a plant pathogenic fungus inhibitory amount of a KP4 polypeptide. The transgenic plant obtained by this method can be a monocot plant or a dicot plant. Transgenic monocot plants of the invention can be selected from the group consisting of barley, maize, corn, flax, oat, rice, rye, sorghum, turf grass, sugarcane, and wheat. Transgenic dicot plants of the invention can be selected from the group consisting of alfalfa, Arabidopsis, barrel medic, banana, broccoli, bean, cabbage, canola, carrot, cassava, cauliflower, celery, citrus, cotton, a cucurbit, eucalyptus, garlic, grape, onion, lettuce, pea, peanut, pepper, potato, poplar, pine, sunflower, safflower, soybean, strawberry, sugar beet, sweet potato, tobacco, and tomato. In certain embodiments, a plant pathogenic fungus inhibitory amount of mature KP4 polypeptide is at least about 0.05 PPM, at least about 0.5 PPM, at least about 1.0 PPM, or at least about 2.0 PPM, where PPM are “parts per million” of KP4 protein present in fresh weight plant tissue. In different embodiments, the growth of a variety of plant pathogenic fungi is inhibited in the transgenic plants of the invention. In different aspects plant pathogenic fungus inhibited by the method can be from the group consisting of an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Macrophomina sp., a Nectria sp., a Peronospora sp., a Phakopsora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp. a Venturia sp., and a Verticillium sp.

In some embodiments, the invention includes transgenic plants comprising a recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence, wherein said transgenic plant expresses said KP4 protein in the apoplast and provides for at least 50% inhibition of a plant pathogenic fungal infection relative to a control plant that lacks said recombinant nucleic acid construct . In different embodiments, the transgenic plant provides for at least 75%, at least 85%, or at least 95% inhibition of a plant pathogenic fungal infection relative to a control plant that lacks said recombinant nucleic acid construct. In certain embodiments, the transgenic plant provides for at least 50% inhibition of a biotrophic plant pathogenic fungus and/or at least 50% inhibition of a necrotrophic plant pathogenic fungus. In certain embodiments, the biotrophic plant pathogenic fungus that is inhibited by the transgenic plants is selected from the group consisting of Ustilago species, Podosphaera species, Erysiphe species, Phakopsora species, and Puccinia species. In certain embodiments, the necrotrophic plant pathogenic fungus that is inhibited by the transgenic plants is selected from the group consisting of Alternaria species, Botrytis species, Colletotrichum species, Cercospora species, Fusarium species, Phoma species, Phytophthora species, Pythium species, Sclerotinia species, and Verticillium species. In certain embodiments, the transgenic plant is a monocot plant or a dicot plant and the non-native nucleic acid sequence comprises one or more non-native codons that are more abundant in monocot plant genes and/or one or more non-native codons that are more abundant in dicot plant genes. In certain embodiments where the plant is a monocot plant, the monocot plant can be selected from the group consisting of barley, maize, corn, flax, oat, rice, rye, sorghum, turf grass, sugarcane, and wheat. In certain embodiments where the plant is a dicot plant, the dicot plant can be selected from the group consisting of alfalfa, Arabidopsis, barrel medic, banana, broccoli, bean, cabbage, canola, carrot, cassava, cauliflower, celery, citrus, cotton, a cucurbit, eucalyptus, garlic, grape, onion, lettuce, pea, peanut, pepper, potato, poplar, pine, sunflower, safflower, soybean, strawberry, sugar beet, sweet potato, tobacco, and tomato. In any of the aforementioned embodiments, the transgenic plants can further comprise a second recombinant nucleic acid construct that provides for expression of MsDef1, MtDef2, MtDef4, Rs-AFP1, or Rs-AFP2. In any of the aforementioned embodiments, the heterologous signal peptide can be a signal peptide of a plant gene. In certain embodiments where the heterologous signal peptide is from a plant gene, the plant gene can be a dicot or a monocot plant gene.

In some embodiments the signal peptide can be from a defensin gene. In one aspect the signal peptide can be from the plant defensin, MsDef . In one aspect the signal peptide has an amino acid sequence at least 80% sequence identity to SEQ. ID. No. 6.

Also provided herein are transgenic plant cells obtained from any of the aforementioned transgenic plants of the invention. Also provided herein are transgenic plant cells comprising any of the DNA constructs of the invention. In some embodiments the transgenic plant comprises a heterologous recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence, wherein said transgenic plant cells express said KP4 protein in the apoplast and provides for at least about 50% inhibition of a plant pathogenic fungal infection relative to a control plant cell that lacks said recombinant nucleic acid construct. In certain embodiments, the transgenic plant cells are obtained by a microbially mediated transformation process including but not limited to Agrobacterium-mediated, Rhizobium- mediated, or Sinorhizobium-mediated transformation.

Also provided herein are transgenic plant seeds obtained from any of the aforementioned transgenic plants or from any of the aforementioned plant cells.

Also provided herein are processed food or feed compositions obtained from either: i) any of the aforementioned the transgenic plant seeds or transgenic plant cells of the invention; or, ii) a transgenic plant part selected from the group consisting of a leaf, a stem, a flower, a root, and a tuber obtained from any of the aforementioned transgenic plants or transgenic plant cells of the invention. In certain embodiments, the processed food or feed composition is a meal, a flour, an oil, or a starch. In certain embodiments, food or feed compositions of the invention comprise a recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence. In certain embodiments, mycotoxin levels in the food or feed composition of the invention are reduced by at least 50% relative to processed food or feed composition that lacks said recombinant nucleic acid construct. In certain embodiments, mycotoxin levels in said food or feed composition of the invention are reduced by at least about 75%, at least about 85%, or at least about 95% relative to processed food or feed composition that lacks said recombinant nucleic acid construct. In certain embodiments, the mycotoxin that is reduced in the food or feed composition of the invention is an aflatoxin, a fumonisin, a vomitoxin, or a trichothecene.

Also provided herein are methods of making a transgenic plant of the invention. In certain embodiments, methods of producing transgenic plants that, comprise the steps of: a) introducing any of the DNA constructs of the invention into a plant. In some embodiments, the DNA construct comprises a recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence into a plant, a plant cell, or a plant tissue; and b) selecting for a transgenic plant comprising said recombinant nucleic acid construct, wherein said transgenic plant selected in step (b) plant expresses said KP4 antifungal protein in the apoplast and provides for at least 50% inhibition of a plant pathogenic fungal infection relative to a control plant that lacks said recombinant nucleic acid construct, are provided. In certain embodiments, the methods provide for a transgenic plant that is a monocot plant or a dicot plant. In certain embodiments, the nucleic acid construct is introduced into said plant, a plant cell or a plant tissue in step (a) by a method selected from the group consisting of particle bombardment, DNA transfection, DNA electroporation, Agrobacterium-mediated, Rhizobium-mediated, and Sinorhizobium-mediated transformation. In certain embodiments, the nucleic acid construct can further comprise a sequence encoding a selectable marker and wherein said transgenic plant is obtained in step (b) by growing said plant, plant cell, or plant tissue under conditions requiring expression of said selectable marker for plant growth. In certain embodiments of the methods, the plant pathogenic fungus is selected from the group consisting of an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Magnaporthe grisea, Macrophomina sp.. a Nectria sp., a Peronospora sp., Phakopsora pachyrhizi, Phialophora gregata, a Phoma sp., a Phymatotrichum sp., a Phytophthora sp., a Plasmopara sp.. a Puccinia sp., a Podosphaera sp., a Pyrenophora sp., a Pyricularia sp, a Pythium sp., a Rhizoctonia sp., a Scerotium sp., a Sclerotinia sp., a Septoria sp., Stenocarpella maydis, a Thielaviopsis sp., an Uncinula sp, a Venturia sp., and a Verticillium sp. In other embodiments, the methods provide for transgenic plants that provide for at least about 75%, at least about 85%, or at least about 95% inhibition of a plant pathogenic fungal infection relative to a control plant that lacks said recombinant nucleic acid construct.

Also provide herewith are methods of obtaining transgenic seed of the invention. In certain embodiments, methods for obtaining transgenic seed can comprise the steps of: i) crossing a transgenic plant comprising a recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence, wherein said transgenic plant expresses said KP4 protein in the apoplast and provides for at least 50% inhibition of a plant pathogenic fungal infection relative to a control plant that lacks said recombinant nucleic acid construct to a plant that lacks said recombinant nucleic acid construct; and, ii) harvesting seed from a pollen recipient from said cross of step (i), are provided. In certain embodiments, the methods can further comprise the steps of iii) selecting for said transgenic seed from said harvested seed; and/or iv) screening for said transgenic seed from said harvested seed.

Also provided are methods for selecting a plant that is resistant to a pathogenic fungal infection. In certain embodiments, such methods of selecting a plant resistant to a pathogenic fungal infection, can comprise the steps of: (i) exposing any of the aforementioned transgenic plants of the invention to a plant pathogenic fungus; and, (ii) obtaining a transgenic plant that exhibits at least 50% inhibition of a plant pathogenic fungal infection relative to a control plant that lacks said recombinant nucleic acid construct. In certain embodiments of the methods, the plant pathogenic fungus is selected from the group consisting of an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Magnaporthe grisea, Macrophomina sp., a Nectria sp., a Peronospora sp., Phakopsora pachyrhizi, Phialophora gregata, a Phoma sp., a Phymatotrichn sp., a Phytophthora sp., a Plasrnopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., Stenocarpella maydis, a Thielaviopsis sp., an Uncinula sp, a Venturia sp., and a Verticillium sp. In certain embodiments of the methods, the transgenic plant provides for at least about 75%, at least about 85%, or at least about 95% inhibition of a plant pathogenic fungal infection relative to a control plant that lacks said recombinant nucleic acid construct. In certain embodiments, the methods can further comprise the step of harvesting at least one plant part selected from the group consisting of a leaf, a stem, a flower, a root, a tuber, or a seed from said plant obtained in step (ii). In certain embodiments of the methods, mycotoxin levels in the harvested plant part are reduced by at least about 50%, at least about 75%, at least about 85%, or at least about 95% relative to a harvested plant part obtained from a control plant that lacks said recombinant nucleic acid construct. In certain embodiments of the methods, the methods can further comprise the step of obtaining a processed food or feed composition from the harvested plant part. In certain embodiments of the methods, the mycotoxin levels in said processed food or feed composition from the harvested plant part are reduced by at least about 50%, at least about 75%, at least about 85%, or at least about 95% relative to a processed food or feed composition obtained from a harvested plant part of a control plant that lacks said recombinant nucleic acid construct.

Also provided herein are transgenic maize plants comprising a chromosome containing an insertion of a KP4 protein expression cassette, wherein said insertion provides for at least 50% inhibition of a plant pathogenic fungal infection relative to a control maize plant that lacks said recombinant nucleic acid construct. In certain embodiments, the transgenic maize plant is selected from the group consisting of transgenic maize plant lines 851, 947, 1040, 885, and 8510. In certain embodiments, the expression plasmid is pZP212-KP4. In some aspects the KP4 expression cassette comprises a nucleic acid sequence which encodes a chimeric protein at least 80% identical to SEQ. ID. No. 1. In some aspects, the KP4 expression cassette comprises a nucleic acid sequence which encodes a mature KP4 protein at least 80% identical to SEQ. ID. No. 4.

Also provided herein are transgenic maize plants comprising a chromosome containing an insertion of a KP4 protein expression cassette, wherein said insertion provides for at least 50% inhibition of a plant pathogenic fungal infection relative to a control maize plant that lacks said recombinant nucleic acid construct. In certain embodiments, the transgenic maize plant is selected from the group consisting of transgenic maize plant lines 851, 947, 1040, 885, and 8510. In certain embodiments, the expression plasmid is pZP212-KP4. In some aspects the KP4 expression cassette comprises a nucleic acid sequence which encodes a protein at least 80% identical to SEQ. ID. No. 1. In some aspects, the KP4 expression cassette comprises a nucleic acid sequence which encodes a protein at least 80% identical to SEQ. ID. No. 4.

Also provided herein are transgenic soybean plants comprising a chromosome containing an insertion of a KP4 protein expression cassette, wherein said insertion provides for at least 50% inhibition of a plant pathogenic fungal infection relative to a control soybean plant that lacks said recombinant nucleic acid construct. In certain embodiments, the protein expression cassette comprises the FMV promoter. In some aspects the KP4 the protein expression cassette comprises a nucleic acid sequence which encodes a chimeric protein at least 80% identical to SEQ. ID. No. 1. In some aspects, the KP4 expression cassette comprises a nucleic acid sequence which encodes a mature KP4 protein at least 80% identical to SEQ. ID. No. 4.

In certain embodiments, the transgenic soybean plant is selected from the group consisting of transgenic soybean plant lines 2, 3, 7, 9, 10, 14, and 16.

### BRIEF DESCRIPTION OF THE DRAWINGS

The description refers to the accompanying drawings in which like references refer to like parts throughout the several views in which:

FIG. 1A shows the nucleic acid sequence of SEQ ID NO:1 that encodes the proprotein of SEQ ID NO:2, where a heterologous signal peptide from the MsDef-1 gene is operably linked to a codon optimized KP4 gene encoded by a non-native DNA sequence.

FIG. 1B shows a schematic diagram of the pPZP212-KP4 vector where a Ubiquitin 1 promoter, a Ubiquitin 1 intron, a TEV 5′ untranslated leader sequence, an MsDef1 signal peptide encoding sequence, a non-native KP4 encoding sequence, and a terminator (i.e. polyadenylation sequence) are all operably linked.

FIG. 1C) Maize leaf material strongly inhibits growth of U. maydis. Approximately 100 mg of fresh leaf tissue was crushed and mixed with lml of PBS buffer. 20 μl of this solution was placed into wells punched into solid agar containing a KP4 sensitive strain of U. maydis (P2 strain). KP4 killing activity is denoted by the clearing zone around the well. As a control, the null segregant, BC1F4, is also shown.

FIG. 2: KP4 transgenic plants resist U. maydis infection. Wild type maize plants (BC1F4) at 10 dpi show disease symptoms of anthocyanin and chlorosis (A), leaf galls (B), and basal galls (C). Wild type maize seedlings 21 days post inoculation of their stems become dead (white arrow 1 in panel D) while the KP4 transgenic plants remain uninfected (line 947; white arrow 2 in panel D). Three-month-old transgenic maize plants (line 947) expressing KP4 protein have no observable developmental differences in comparison to the wild-type (BC1F4; panels E, F). Maize ears prior to U. maydis inoculation (panels G,H). Galls appear 14 days post inoculation in the wild-type (arrow 3 in panel I), while the KP4 transgenic maize remains free of disease for the duration of the experiment (J).

FIGS. 3A and 3B: Expression of bioactive KP4 in transgenic soybean. Wild type represents a leaf tissue extract from a non-transgenic control soybean plant that does not express KP4. The wells labelled KP4 contained a positive control sample of KP4 protein.

FIG. 4A and 4B shows exemplary DNA constructs comprising expression cassettes for the expression of the chimeric KP4 proteins of the invention. FIG. 4A shows a DNA construct suitable for the expression of the chimeric KP4 proteins of the invention in Maize. In FIG. 4A, the abbreviation “LB”=T-DNA left border; “T-DNA RB”=T-DNA right border; “Ter”=Cauliflower mosaic virus 35 S terminator sequence; “nptll”=Neomycin phosphotransferase II; “35”=Cauliflower mosaic virus 35S promoter; “Ubi and Ubi intron”=Maize ubiquitin promoter and intron; “TEV”=Tobacco etch virus mRNA translational enhancer; “Ori”=Origin of replication; “TraF”=Transfer F; “Tet(R)”=tetracycline resistance gene; “Kan(R)=Kanamycin resistance gene; “TrfA”=T-DNA replication factor, and “KP4” represents the chimeric KP4 protein (SEQ. ID. No 1) of the invention.

FIG. 4B shows a DNA construct suitable for the expression of the chimeric KP4 proteins of the invention in soybean. In FIG. 4 B, the abbreviation “LB”=T-DNA left border; “T-DNA RB”=T-DNA right border; “tNOS”=Nopaline synthase terminator sequence; ; “nptll”=Neomycin phosphotransferase II; “35”=Cauliflower mosaic virus 35S promoter; “FMV”=Figwort mosaic virus 35S; “SU intron”=Super ubiquitin intron; “BAR”=Bialophos resistance gene; “NOS”=Nopaline synthase promoter; “Ori”=Origin of replication; “TraF”=Transfer F; “Tet(R)”=tetracycline resistance gene; “Kan(R) ”=Kanamycin resistance gene; “TrfA”=T-DNA replication factor, and “KP4” represents the chimeric KP4 protein (SEQ. ID. No 1) of the invention.

### DETAILED DESCRIPTION OF THE INVENTION

Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

The phrases “antifungal polypeptide” or “antifungal protein” as used herein refer to polypeptides or proteins which exhibit any one or more of the following characteristics; inhibiting the growth of fungal cells, killing fungal cells, disrupting or retarding stages of the fungal life cycle such as spore germination, sporulation, or mating, and/or disrupting fungal cell infection, penetration or spread within a plant.

The phrase “biological functional equivalents” as used herein refers to peptides, polypeptides and proteins that contain a sequence or structural feature similar to a KP4 protein of the present invention, and which exhibit the same or similar (i.e. at least 50%, or at least 75%, or at least 80% of the antifungal activity of a KP4 protein of the present invention, in one of the antifungal assays as described in the Examples.

The phrases “combating fungal damage”, “combating or controlling fungal damage” or “controlling fungal damage” as used herein refer to reduction (i.e. at least a 10% decrease, at least a 20% decrease, at least a 30% decrease, or at least a 50% or greater decrease in damage compared to a control wild type plant) in damage to a crop plant or crop plant product due to infection by a fungal pathogen. More generally, these phrases refer to reduction in the adverse effects caused by the presence of an undesired fungus in the crop plant. Adverse effects of fungal growth are understood to include any type of plant tissue damage or necrosis, any type of plant yield reduction, any reduction in the value of the crop plant product, and/or production of undesirable fungal metabolites or fungal growth by-products including but not limited to mycotoxins.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged / polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged / polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free -OH can be maintained; and Gln for Asn such that a free -NH₂ can be maintained.

The phrase “DNA construct” as used herein refers to any DNA molecule in which two or more ordinarily distinct DNA sequences have been covalently linked. Examples of DNA constructs include but not limited to plasmids, cosmids, viruses, BACs (bacterial artificial chromosome), YACs (yeast artificial chromosome), plant minichromosomes, autonomously replicating sequences, phage, or linear or circular single-stranded or double-stranded DNA sequences, derived from any source, that are capable of genomic integration or autonomous replication. DNA constructs can be assembled by a variety of methods including but not limited to recombinant DNA techniques, DNA synthesis techniques, PCR (Polymerase Chain Reaction) techniques, or any combination of techniques.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example. mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, that have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise expression control sequences (i.e., non-coding) sequences as well as coding sequences and introns. The transcribed region of the gene may also include untranslated regions including introns, a 5′-untranslated region (5′-UTR) and a 3′-untranslated region (3′-UTR).

The term “heterologous” refers to a nucleic acid or protein which has been introduced into an organism (such as a plant, animal, or prokaryotic cell), or a nucleic acid molecule (such as chromosome, vector, or nucleic acid construct), which are derived from another source, or which are from the same source, but are located in a different (i.e. non native) sequence context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention.

To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., (1987) Cell, 50:667). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The phrase “a plant pathogenic fungus inhibitory amount”, as used herein in the context of a transgenic plant expressing a KP4 polypeptide, refers to an amount of a KP4 polypeptide that results in any measurable decrease (i.e. at least a 10% decrease, at least a 20% decrease, at least a 30% decrease, at least a 40% decrease, at least a 50% decrease or greater compared to a wild type plant exposed to the fungus under the same conditions) in fungal growth in the transgenic plant and/or any measurable decrease in the adverse effects caused by fungal growth in the transgenic plant.

The phrases “more abundant in monocot plant genes” and “more abundant in dicot plant genes” as used herein in reference to codon usage in a gene refers to codons that occur at a higher frequency in monocot and/or dicot plant genes than other codons that encode the same amino acid in monocot and/or dicot plant genes.

The phrase “inhibiting growth of a plant pathogenic fungus” as used herein refers to methods that result in any measurable decrease (i.e. at least a 10% decrease, at least a 20% decrease, at least a 30% decrease, at least a 40% decrease, at least a 50% decrease or greater compared to a wild type plant exposed to the fungus under the same conditions) in fungal growth, where fungal growth includes but is not limited to any measurable decrease in the numbers an/or extent of fungal cells, spores, conidia, or mycelia. As used herein, “inhibiting growth of a plant pathogenic fungus” is also understood to include any measurable decrease in the adverse effects cause by fungal growth in a plant. Adverse effects of fungal growth in a plant include any type of plant tissue damage or necrosis, any type of plant yield reduction, any reduction in the value of the crop plant product, and/or production of undesirable fungal metabolites or fungal growth by-products including but not limited to mycotoxins.

The terms “operably linked”, “operatively linked,” or “operatively coupled” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more DNA or RNA nucleotide sequences including, but not limited to: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polypeptide” and “protein” are used interchangeably herein to refer to a polymer of amino acid residues and to variants and synthetic and naturally occurring analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers and naturally occurring chemical derivatives thereof. Such derivatives include, for example, post-translational modifications and degradation products including pyroglutamyl, iso-aspartyl, proteolytic, phosphorylated, glycosylated, oxidatized, isomerized, and deaminated variants of KP4

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the -10 and -35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), the cassava vein mosaic virus (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence,” “comparison window,” “sequence identity,” “percentage of sequence identity” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology,” John Wiley & Sons Inc, 1994-1998, Chapter 15.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes).

In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap. which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frame shift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Similarly, in particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

The term “regeneration” as used herein refers to any method of obtaining a whole plant from any one of a seed, a plant cell, a group of plant cells, plant callus tissue, or an excised piece of a plant.

The term “transformation” as used herein refers to a process of introducing an exogenous DNA sequence (e.g., a vector, a recombinant DNA molecule) into a cell or protoplast in which that exogenous DNA is incorporated into a chromosome or is capable of autonomous replication. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction, or infection with viruses or other infectious agents.

“Transformed”, “transduced”, or “transgenic”, in the context of a cell or plant, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells or plants have been through the transformation process and contain foreign nucleic acid. The term “untransformed” refers to cells that have not been through the transformation process.

The term “vector” as used herein refers to a DNA or RNA molecule capable of replication in a host cell and/or to which another DNA or RNA segment can be operatively linked so as to bring about replication of the attached segment. A plasmid is an exemplary vector.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Miki and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Exemplary KP4 Proteins

In any of these methods, fusion proteins, DNA constructs, and transgenic organisms, the terms “KP4”, or “KP4 protein”, or “KP4 polypeptide” or “KP4 antifungal protein” refers to all naturally-occurring and synthetic forms of KP4 that retain anti-fungal activity, for example as determined using any of the methods described in Examples 2 or 3. In one aspect the KP4 protein is from a UMV4 virus. Representative species and Gene bank accession numbers for various KP4 genes are listed below in Table Dl. The terms “mature KP4 polypeptide” or “mature KP4 protein” refers to a KP4 protein lacking its native signal peptide. An exemplary mature KP4 is provided in SEQ. ID. No. 4.

TABLE D1

| | | GenBank |
|---|---|---|
| | | Accession |
| | | No. and/ |
| Organism/ | | or SEQ ID |
| Description | Sequence | NO. |
| Ustilago | MQIINVVYSF LFAAAMLPVV HSLGINCRGS | Q90121.1 |
| maydis P4 | SQCGLSGGNL MVRIRDQACG NQGQTWCPGE | SEQ. ID. |
| virus (UmV4) | RRAKVCGTGN SISAYVQSTN NCISGTEACR | No. 7 |
| (UmV-P4) | HLTNLVNHGC RVCGSDPLYA GNDVSRGQLT | |
| Complete | VNYVNSC | |
| protein with | | |
| native signal | | |
| sequence | | |
| Mature protein | LGINCRGS SQCGLSGGNL MVRIRDQACG NQGQTWCPGE | SEQ. ID. |
| lacking signal | RRAKVCGTGN SISAYVQSTN NCISGTEACR | No. 4 |
| sequence | HLTNLVNHGC RVCGSDPLYA GNDVSRGQLT VNYVNSC | |

Nucleic Acid Sequences

| | | |
|---|---|---|
| Ustilago | ATGCAGATTATAAATGTGGTATACTCCTTCCTGTTTGCGGCAG | U25179.1 |
| maydis P4 | CAATGCTTCCAGTGGTTCACTCCTTAGGGATTAATTGTAGGGG | SEQ. ID. |
| virus | CAGCTCGCAATGTGGGTTATCCGGCGGGAACCTTATGGTCCGA | No. 8 |
| (UmV4) | ATAAGAGATCAGGCATGTGGTAACCAAGGCCAAACATGGTGTC | |
| (UmV-P4) | CTGGCGAACGGCGTGCTAAGGTCTGTGGGACTGGCAATAGCAT | |
| complete | CTCTGCTTATGTTCAGTCTACCAACAATTGTATATCAGGGACA | |
| cDNA | GAGGCCTGTCGCCATTTGACTAACCTCGTTAACCATGGTTGTA | |
| sequence | GAGTCTGTGGCAGCGACCCACTCTATGCCGGGAATGATGTGTC | |
| | CCGAGGGCAGTTGACTGTCAACTACGTAAACTCGTGTTGATAC | |
| | GGACCACTATCGAAGTGTGTGGTATATGGTAGTAGGACTTGAG | |
| | GGGTTATAGACGCCAACGGTGGGGACACTGTAGGGGAAACTGG | |
| | TGTA | |
| Mature | TTAGGGATTAATTGTAGGGGCAGCTCGCAATGTGGGTTATCCG | SEQ. ID. |
| sequence | GCGGGAACCTTATGGTCCGAATAAGAGATCAGGCATGTGGTAA | No. 9 |
| (native | CCAAGGCCAAACATGGTGTCCTGGCGAACGGCGTGCTAAGGTC | |
| codon | TGTGGGACTGGCAATAGCATCTCTGCTTATGTTCAGTCTACCA | |
| usage) | ACAATTGTATATCAGGGACAGAGGCCTGTCGCCATTTGACTAA | |
| | CCTCGTTAACCATGGTTGTAGAGTCTGTGGCAGCGACCCACTC | |
| | TATGCCGGGAATGATGTGTCCCGAGGGCAGTTGACTGTCAACT | |
| | ACGTAAACTCGTGTTGATACGGACCACTATCGAAGTGTGTGGT | |
| | ATATGGTAGTAGGACTTGAGGGGTTATAGACGCCAACGGTGGG | |
| | GACACTGTAGGGGAAACTGGTGTA | |
| Codon | CTCGGCATCAACTGCCGCGGCTCCAGCCAGTGCGGCCTCTCCG | SEQ. ID. |
| optimized | GCGGCAACCTGATGGTGAGGATCAGGGACCAGGCCTGCGGCAA | No. 3 |
| for enhanced | CCAGGGCCAGACCTGGTGCCCAGGCGAGAGGAGGGCCAAGGTG | |
| monocot | TGCGGCACCGGCAACAGCATCAGCGCCTACGTGCAGAGCACCA | |
| expression | ACAACTGCATCAGCGGCACCGAGGCCTGCCGCCACCTCACCAA | |
| | CCTCGTGAACCACGGCTGCCGCGTGTGCGGCAGCGACCCGCTG | |
| | TACGCCGGCAACGACGTGTCCAGGGGCCAGCTCACCGTGAACT | |
| | ACGTGAACAGCTGC | |

Preferably the KP4 protein which may be used in any of the methods, chimeric proteins, DNA constructs, and plants of the invention may have an amino acid sequence which is substantially homologous, or substantially similar to any of the KP4 sequences, for example, to any of the native or synthetic amino acid sequences listed in Table Dl.

Alternatively, the KP4 may have an amino acid sequence having at least 30% preferably at least 40, 50, 60, 70, 75, 80, 85, 90, 95, 98, or 99% identity with a KP4 listed in Table Dl. In certain embodiments, the KP4 protein for use in any of the methods and plants of the present invention is at least 80% identical to the mature KP4 from Ustilago maydis P4 virus (UmV4) (UmV-P4) (SEQ. ID. No. 4).

### KP4 Polynucleotides

Certain embodiments relate to polynucleotides that encode a KP4 protein. Among other uses, these embodiments may be utilized to recombinantly produce a desired KP4 protein or variant thereof, or to express the KP4 protein in a selected cell or plant. It will be appreciated by those of ordinary skill in the art that, as a result of the degeneracy of the genetic code, there are many nucleotide sequences that encode a KP4 protein as described herein. Some of these polynucleotides may bear minimal homology to the nucleotide sequence of any native gene. Nonetheless, polynucleotides that vary due to differences in codon usage are specifically contemplated by the present invention, for example polynucleotides that are optimized for monocot, dicot, yeast, or bacterial codon selection.

Therefore, multiple polynucleotides can encode the KP4 proteins of the invention. Moreover, the polynucleotide sequence can be manipulated for various reasons. Examples include but are not limited to the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, decrease the density of CpG dinucleotide motifs (see for example, Kameda et al., Biochem. Biophys. Res. Commun (2006) 349(4): 1269-1277) or reduce the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (Calif., USA).

In general, non-native nucleic acids that encode KP4 proteins can be obtained from synthetic KP4 genes derived by “back-translation” of KP4 polypeptide sequences, from genomic clones, from deduced coding sequences derived from KP4 genomic clones, from cDNA or EST sequences, and from any of the foregoing sequences that have been subjected to mutagenesis. Examples of nucleic acids that contain mature KP4 protein-encoding nucleotide sequences include but are not limited to a sequence with at least 70%, at least 80%, at least 85%, at least 90%, at least 95%, at least 97%, at least 99%, or 100% sequence identity to SEQ. ID. NO: 3, or SEQ. ID. No.9.

In other embodiments of the invention, the KP4-encoding gene can be synthesized de novo from a KP4 mature peptide sequence. The sequence of the KP4 gene can be deduced from the KP4 peptide sequence through use of the genetic code. Computer programs such as “BackTranslate” (GCGTM Package, Acclerys, Inc. San Diego, Calif.) can be used to convert a peptide sequence to the corresponding nucleotide sequence that encodes the peptide. Examples of KP4 protein sequences that can be used to obtain corresponding nucleotide encoding sequences include, but are not limited to, KP4 mature peptide sequences such as SEQ ID NO: 4 and the biological functional equivalents of that amino acid sequences. Biological functional equivalents of a KP4 protein can also include mature KP4 polypeptides with at least 70%, at least 80%, at least 85%, at least 90%, at least 95%, or 100% sequence identity SEQ ID NO: 4.

Furthermore, the non-native KP4—encoding nucleotide sequence can designed so that it will be expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the non-native KP4—encoding nucleotide sequence are codons that are more abundant in monocot and/or dicot plant genes. Codon usage in various monocot or dicot genes has been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the non-native KP4—encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the KP4 polynucleotide sequences listed in Table D1. Polynucleotide “variants” may contain one or more substitutions, additions, deletions and/or insertions in relation to a reference polynucleotide. Generally, variants of the KP reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (i.e. to any of SEQ. ID, No. 3, SEQ. ID. No. 8 or SEQ, ID. No. 9) as determined by sequence alignment programs described elsewhere herein using default parameters.

The KP4 may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions. Exemplary naturally occurring isoforms of KP4 include for example forms comprising the amino acid substitutions W56I, C66E, G67C, S73D, A74H and T79S

Naturally-occurring chemical modifications including post-translational modifications and degradation products of the KP4 are also specifically included in any of the methods of the invention including for example, pyroglutamyl, iso-aspartyl, proteolytic, phosphorylated, glycosylated, reduced, oxidatized, isomerized, and deaminated variants of the KP4.

A variety of DNA sequences encoding a variety of mature KP4 proteins can be used in practicing this invention. The DNA sequence can encode mature KP4 proteins (i.e. proteins lacking the native signal peptide) that include, but are not limited to, the biological functional equivalents of any of the foregoing amino acid sequences. Biological functional equivalents of a KP4 protein also include, but are not limited to, mature KP4 polypeptides with at least 70%, at least 80%, at least 85%, at least 90%, at least 95%, or 100% sequence identity to SEQ ID NO: 4.

Chimeric KP4 Genes

Certain embodiments of this invention comprise a sequence encoding a heterologous signal peptide that allows for secretion of the mature KP4 protein from the cells. Such signal peptide sequences can include synthetic, or naturally occurring, signal peptide sequences derived from other well characterized secreted proteins which are joined to the coding sequence of an expressed gene, and are removed post-translationally from the initial translation product.

In certain embodiments, the heterologous signal peptide sequences derived from Medicago defensin proteins (Hanks et al, 2005) can be used. Examples of Medicago defensin protein signal peptides include, but are not limited to, signal peptides of MsDef1, (GenBank Accession No. AAV85437.1), MtDef1.1, (GenBank Accession No AAQ91287.1) MsDef1.6 (GenBank Accession No AAV85432.1), MtDef4, Sagaram et al., (2011) PLoS One. 6(4):e18550), and MtDef2.1 (GenBank Accession No AAQ91290.1). The MtDef4 signal peptide and sequences encoding the same are disclosed in US Patent Application Publication No. 20080201800. Another example of a useful heterologous signal peptide encoding sequence that can be used in monocot plants is the signal peptide derived from a barley cysteine endoproteinase gene (Koehler and Ho, 1990). Another example of a useful heterologous signal peptide encoding sequence that can be used in dicot plants is the tobacco PR1b signal peptide. It is understood that this group of exemplary heterologous signal peptides is non-limiting and that one skilled in the art could employ other heterologous signal peptides that are not explicitly cited here in the practice of this invention.

In certain embodiments of the invention, the heterologous signal peptide in such chimeric constructs may have an amino acid sequence having at least 30% preferably at least 40, 50, 60, 70, 75, 80, 85, 90, 95, 98, or 99% identity with a signal peptide from MsDef1 (SEQ. ID. No. 6). In certain embodiments, the signal peptide for use in any of the methods and plants of the present invention is at least 80% identical to the signal peptide from MsDef1 (SEQ. ID. No. 6).

In certain embodiments of the invention, the heterologous signal peptide in such chimeric constructs provides for secretion of the mature KP4 protein to the apoplast. KP4 polypeptides that are operably linked to a heterologous signal peptide are expected to enter the secretion pathway and accumulate in the apoplast.

A non-limiting example of a synthetic nucleotide sequence optimized for expression in monocot plants is SEQ ID NO: 1. The chimeric gene represented by SEQ ID NO: 1 encodes a proprotein (SEQ ID NO: 2) comprising a MsDef1 signal peptide (SEQ ID NO: 6) that is operably linked to a KP4 mature peptide sequence (SEQ ID NO: 4).

In certain embodiments, the chimeric KP4 gene for use in any of the methods and plants of the present invention may have at least about 30%, 40% 50%, 55%, 60%, 65%, 70%, generally at least about 75%, 80%, 85%, desirably about 90% to 95% or more, and more suitably about 98% or more sequence identity to SEQ, ID. No. 1 as determined by sequence alignment programs described elsewhere herein using default parameters.

In other embodiments of the invention, additional sequences encoding peptides that provide for the localization of a KP4 protein in subcellular organelles can be operably linked to the KP4 polypeptide. KP4 polypeptides that are operably linked to a heterologous signal peptide are expected to enter the secretion pathway and can be retained by organelles such as the endoplasmic reticulum (ER) or targeted to the vacuole by operably linking the appropriate retention or targeting peptides to the C-terminus of the KP4 polypeptide. Examples of vacuolar targeting peptides include, but are not limited to a CTPP vacuolar targeting signal from the barley lectin gene. Examples of ER targeting peptides include, but are not limited to a peptide comprising a KDEL amino acid sequence. Without seeking to be limited by theory, localization of KP4 polypeptides in either the endoplasmic reticulum or the vacuole can provide for desirable properties such as increased expression in transgenic plants and /or increased efficacy in inhibiting fungal growth in transgenic plants.

### KP4 Proteins

Peptides, polypeptides, and proteins biologically functionally equivalent to KP4 also include, but are not limited to, amino acid sequences containing conservative amino acid substitutions in the mature KP4 protein sequences. Examples of mature KP4 proteins that can be substituted to obtain biological equivalents include, but are not limited to, the KP4 consensus sequence.

In such amino acid sequences, one or more amino acids in the sequence is (are) substituted with another amino acid(s), the charge and polarity of which is similar to that of the native amino acid, i.e., a conservative amino acid substitution, resulting in a silent change.

Substitutes for an amino acid within the KP4 polypeptide sequence can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine.

Conservative amino acid changes within the KP4 polypeptide sequence can be made by substituting one amino acid within one of these groups with another amino acid within the same group. Biologically functional equivalents of KP4 can have 10 or fewer conservative amino acid changes, more preferably seven or fewer conservative amino acid changes, and most preferably five or fewer conservative amino acid changes. The encoding nucleotide sequence (gene, plasmid DNA, cDNA, or synthetic DNA) will thus have corresponding base substitutions, permitting it to encode biologically functional equivalent forms of KP4.

The biologically functional equivalent peptides, polypeptides, and proteins contemplated herein should possess about 70% or greater sequence identity, preferably about 85% or greater sequence identity, and most preferably about 90% to 95% or greater sequence identity, to the sequence of, or corresponding moiety within, the KP4 polypeptide sequence. In certain embodiments of the invention, biologically functional equivalent peptides, polypeptides, and proteins possessing about 80% or greater sequence identity, preferably about 85% or greater sequence identity, and most preferably about 90% to 95% or greater sequence identity, to the sequence of KP4 (SEQ ID NO:4).

As described herein, modification and changes may be made in the structure of the KP4 proteins of the present invention and nucleic acids which encode them and still obtain a functional molecule that encodes a KP4 protein or peptide with desirable characteristics. The following is a discussion based upon changing the amino acids of a protein to create an equivalent, or even an improved, second-generation molecule. In particular embodiments of the invention, mutated KP4 proteins are contemplated to be useful for increasing the antifungal activity of the protein, and consequently increasing the antifungal activity and/or expression of the recombinant transgene in a plant cell. The amino acid changes may be achieved by changing the codons of the DNA sequence, according to the codons given in Table D2.

TABLE D2

| | Amino | |
|---|---|---|
| | Acid | |
| Amino Acids | Codes | Codons |
| Alanine | Ala (A) | GCA GCC GCG GCA |
| Cysteine | Cys (C) | UGC UGU |
| Aspartic acid | Asp (D) | GAC GAU |
| Glutamic acid | Glu (E) | GAA GAG |
| Phenylalanine | Phe (F) | UUC UUU |
| Glycine | Gly (G) | GGA GGC GGG GGU |
| Histidine | His (H) | CAC CAU |
| Isoleucine | Ile (I) | AUA AUC AUU |
| Lysine | Lys (K) | AAA AAG |
| Leucine | Leu (L) | UUA UUG CUA CUC CUG |
| | | CUU |
| Methionine | Met (M) | AUG |
| Asparagine | Asn (N) | AAC AAU |
| Proline | Pro (P) | CCA CCC CCG CCU |
| Glutamine | Gln (Q) | CAA CAG |
| Arginine | Arg (R) | AGA AGG CGA CGC CGG |
| | | CGU |
| Serine | Ser (S) | AGC AGU UCA UCC UCG |
| | | UCU |
| Threonine | Thr (T) | ACA ACC ACG ACU |
| Valine | Val (V) | GUA GUC GUG GUU |
| Tryptophan | Trp (W) | UGG |
| Tyrosine | Tyr (Y) | UAC UAU |

For example, certain amino acids may be substituted for other amino acids in a protein structure without appreciable loss of biochemical or biological activity. Since it is the interactive capacity and nature of a protein that defines that protein's biological functional activity, certain amino acid sequence substitutions can be made in a protein sequence, and, of course, it's underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, 1982). These are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within.±2 is preferred, those which are within±1 are particularly preferred, and those within±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0.+0.1); glutamate (+3.0.+0.1); serine (+0.3); asparagine (+0.2); glutamine (+0.2); glycine (0); threonine (−0.4); proline (−0.5.+0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Non-Conservative Substitutions in the KP4 Polypeptides

It is further recognized that non-conservative substitutions in KP4 polypeptide sequences can be made to obtain KP4 polypeptides that are the functional biological equivalents of the KP4 polypeptides disclosed herein. In these instances, the non-conservative substitutions can simply be tested for inhibition of fungal growth to identify non-conservative substitutions that provide for functional biological equivalents of a given KP4 polypeptide.

### Fragments and Variants of KP4:

While the antifungal polypeptide of the present invention preferably comprises a mature KP4 protein sequence, fragments and variants of this sequence possessing the same or similar antifungal activity as that of this antifungal protein are also encompassed by the present invention. Fragments or variants of KP4 with antifungal activity that are anticipated by this invention can also comprise amino acid substitutions, deletions, insertions or additions in a KP4 protein sequence.

The antifungal polypeptide of the present invention preferably comprises the mature KP4 protein sequence (SEQ ID NO:4), fragments and variants of this sequence possessing the same or similar antifungal activity as that of this particular KP4 protein are also encompassed by the present invention. The fragments or variants with antifungal activity that are anticipated by this invention can also comprise amino acid substitutions, deletions, insertions or additions of the sequence shown in SEQ ID NO: 4.

Fragments of the mature KP4 protein can be truncated forms wherein one or more amino acids are deleted from the N-terminal end, C-terminal end, the middle of the protein, or combinations thereof with antifungal activity are also anticipated by this invention. These fragments can be naturally occurring or synthetic mutants of KP4, and retain the antifungal activity of KP4. A preferred mature form of the KP4 protein that can be used to obtain truncated derivatives with antifungal activity is the mature KP4 protein of SEQ ID NO:4.

Variants of KP4 include forms wherein one or more amino acids has/have been inserted into the natural sequence. These variants can also be naturally occurring or synthetic mutants of KP4, and should retain the antifungal activity of KP4.

Combinations of the foregoing, i.e., forms of the antifungal polypeptide containing both amino acid deletions and additions, are also encompassed by the present invention. Amino acid substitutions can also be present therein as well.

The fragments and variants of KP4 encompassed by the present invention should preferably possess about 70-75% or greater sequence identity, more preferably about 80%, 85%, 88% or greater sequence identity, and most preferably about 90% to 95% or greater amino acid sequence identity, to the corresponding regions of the mature KP4 protein having the corresponding amino acid sequences shown in SEQ ID NO: 4.

Use of Structure Function Relationships to Design KP4 Variants

The structure of the KP4 protein was determined by the Smith laboratory in 1995 at which time the mechanism of action was proposed [8]. This mechanism was deduced from features found at the C-terminus of the protein that was reminiscent of other ion channel blockers. This mode of action was subsequently demonstrated by the Smith lab by the observation that KP4 blocks the uptake of calcium by the target fungus. Further, mutagenesis studies demonstrated that mutations around the C-terminus (i.e. K42Q) abrogated antifungal activity while mutations elsewhere (i.e. R68Q) had no effect on the antifungal activity. It is thus anticipated that KP4 variants comprising mutations in positions that include, but are not limited to R68Q, can be used to in the methods, plants, seeds, and processed plant products of the invention.

Other Biologically Functional Equivalent Forms of KP4

Other biologically functional equivalent forms of KP4 useful in the present invention include conjugates of the polypeptides, or biologically functional equivalents thereof as described above, with other peptides, polypeptides, or proteins, forming fusion products therewith exhibiting the same, similar, or greater antifungal activity as compared with that of KP4 having the amino acid sequence shown in SEQ ID NO:4.

Simultaneous co-expression of multiple antifungal and/or other anti-pathogen proteins in plants is advantageous in that it exploits more than one mode of control of plant pathogens. This may, where two or more antifungal proteins are expressed, minimize the possibility of developing resistant fungal species, broaden the scope of resistance and potentially result in a synergistic antifungal effect, thereby enhancing the level of resistance.

KP4 proteins and biologically functional equivalents are therefore expected to be useful in controlling fungi in a wide variety of plants, exemplified by those in the following genera and species: Alternaria (Alternaria brassicola; Alternaria solani); Ascochyta (Ascochyta pisi); Botrytis (Botrytis cinerea); Cercospora (Cercospora kikuchii; Cercospora zeae-maydis); Colletotrichum (Colletotrichum lindemuthianum); Diplodia (Diplodia maydis); Erysiphe (Erysiphe graminis f.sp. graminis; Erysiphe graminis f.sp. hordei); Fusarium (Fusarium nivale; Fusarium oxysporum; Fusarium graminearum; Fusarium culmorum; Fusarium solani; Fusarium moniliforme; Fusarium roseum); Gaeumanomyces (Gaeumanomyces graminis f.sp. tritici); Helminthosporium (Helminthosporium turcicum; Helminthosporium carbonum; Helminthosporium maydis); Macrophomina (Macrophomina phaseolina; Maganaporthe grisea); Nectria (Nectria heamatococca); Peronospora (Peronospora manshurica; Peronospora tabacina); Phakopsora (Phakopsora pachyrhizi); Phoma (Phoma betae); Phymatotrichum (Phymatotrichum omnivorum); Phytophthora (Phytophthora cinnamomi; Phytophthora cactorum; Phytophthora phaseoli; Phytophthora parasitica; Phytophthora citrophthora; Phytophthora megasperma f.sp. sojae; Phytophthora infestans); Plasmopara (Plasmopara viticola); Podosphaera (Podosphaera leucotricha); Puccinia (Puccinia sorghi; Puccinia striiformis; Puccinia graminis f.sp. tritici; Puccinia asparagi; Puccinia recondita; Puccinia arachidis); Pythium (Pythium aphanidermatum); Pyrenophora (Pyrenophora tritici-repentens); Pyricularia (Pyricularia oryzae); Pythium (Pythium ultimum); Rhizoctonia (Rhizoctonia solani; Rhizoctonia cerealis); Scerotium (Scerotium rolfsii); Sclerotinia (Sclerotinia sclerotiorum); Septoria (Septoria lycopersici; Septoria glycines; Septoria nodorum; Septoria tritici); Thielaviopsis (Thielaviopsis basicola); Uncinula (Uncinula necator); Venturia (Venturia inaequalis); Verticillium (Verticillium dahliae; Verticillium alboatrum). Transgenic plants provided herewith can also be used to control Fusarium graminearum, Fusarium verticillioides and/or F. prohferatum.

### DNA Constructs

In one aspect the DNA constructs and expression vectors for the KP4 proteins comprise nucleic acid sequences encoding any of the previously described mature KP4 proteins as described in Table D1 operatively coupled to an expression control sequences, heterologous signal peptide sequence and transcriptional terminator for efficient expression in the plant of interest.

In one aspect of any of these expression vectors, and DNA constructs the nucleic acid encoding the mature KP4 protein is codon optimized for expression in the plant of interest. In some aspects the nucleic acid encoding the codon optimized mature KP4 is at least 80% identical to SEQ. ID. No. 3. In one aspect the KP4 protein encoded by the nucleic acid has an amino acid sequence which is at least about 80% identical to SEQ. ID. No. 4.

In one aspect of any of these expression vectors, and DNA constructs the heterologous signal peptide sequence is from a plant secreted protein. In some embodiments the signal peptide is from a defensin gene. In some embodiments the signal peptide is codon optimized for expression in the plant of interest. In some aspects the signal peptide is from MsDef1. In some aspects the nucleic acid encoding the codon optimized MsDef1 signal peptide is at least 80% identical to SEQ. ID. No. 5. In one aspect the nucleic acid encoding the signal peptide encodes an amino acid sequence which is at least about 80% identical to SEQ. ID. No. 6.

In one aspect the DNA constructs and expression vectors comprise a nucleic acid sequence at least about 80% identical to SEQ. ID. No. 1.

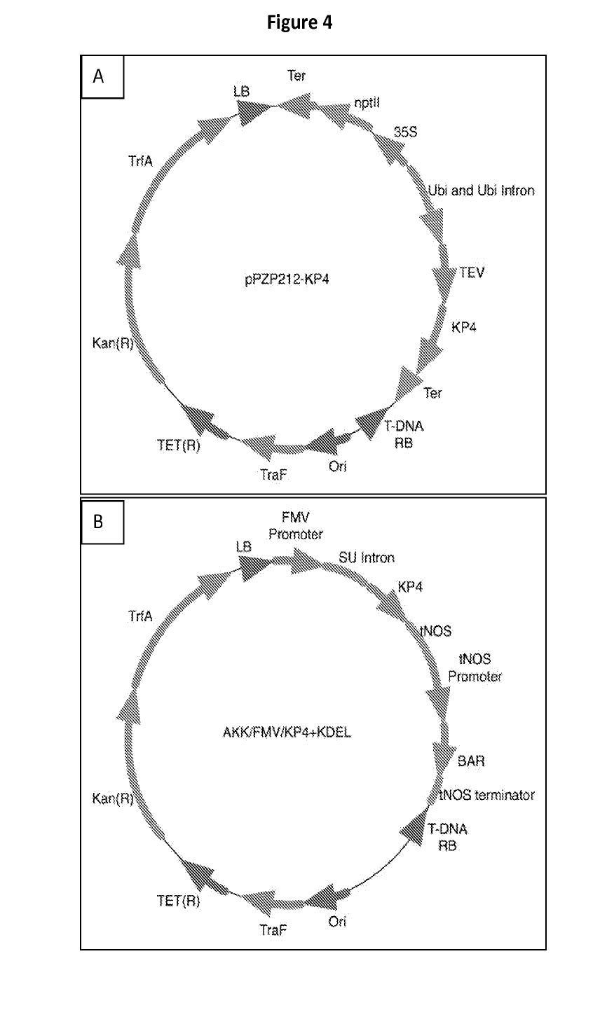

In some embodiments, the DNA constructs and expression vectors of the invention further comprise polynucleotide sequences encoding one or more of the following elements i) a selectable marker gene to enable antibiotic selection, ii) a screenable marker gene to enable visual identification of transformed cells, and iii) T—element DNA sequences to enable Agrobacterium tumefaciens mediated transformation. Exemplary expression cassettes are described in the Examples, and shown schematically in FIGS. 4A and 4B.

In some embodiments the expression vector comprises a vector backbone selected from pBin, pCAMBIA, pCGN, EHA105 and pZP212.

Those of skill in the art will appreciate that the foregoing descriptions of expression cassettes represents only illustrative examples of expression cassettes that could be readily constructed, and is not intended to represent an exhaustive list of all possible DNA constructs or expression cassettes that could be constructed.

Moreover expression vectors suitable for use in expressing the claimed DNA constructs in plants, and methods for their construction are generally well known, and need not be limited. These techniques, including techniques for nucleic acid manipulation of genes such as subcloning a subject promoter, or nucleic acid sequences encoding a gene of interest into expression vectors, labeling probes, DNA hybridization, and the like, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site directed mutagenesis can be used to introduce a restriction site at the start codon of a heterologous gene of interest. Heterologous DNA sequences are then linked to a suitable expression control sequences such that the expression of the gene of interest are regulated (operatively coupled) by the promoter.

DNA constructs comprising an expression cassette for the gene of interest can then be inserted into a variety of expression vectors. Such vectors include expression vectors that are useful in the transformation of plant cells. Many other such vectors useful in the transformation of plant cells can be constructed by the use of recombinant DNA techniques well known to those of skill in the art as described above.

Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein

Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Canberra, Australia); pGA482, An et al, EMBO J., 4:277-284, 1985; pCGN1547, (CALGENE Inc.) McBride et al, Plant Mol. Biol., 14:269-276, 1990, pZP212 (Hajdukiewicz et al., Plant. Mol. Biol. 25 989-994), EHA105 and the like vectors, such as is described herein.

DNA constructs will typically include expression control sequences comprising promoters to drive expression of the KP4 gene within the organism. Promoters may provide ubiquitous, cell type specific, constitutive promoter or inducible promoter expression. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is, regions comprising sequences from the CAAT box to the translational start site, with other upstream promoter elements to enhance or otherwise alter promoter activity or specificity.

In some aspects promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, the maize Ubil promoter / intron/tobacco etch virus mRNA leader sequence, and the heat shock protein 70 gene of Petunia hybrida (U.S. Pat. No. 5,659,122).

Depending upon the host cell system utilized, any one of a number of suitable promoters can be used. Promoter selection can be based on expression profile and expression level. One broad class of useful promoters are referred to as “constitutive” promoters in that they are active in most plant organs throughout plant development. For example, the promoter can be a viral promoter such as a CaMV35S or FMV35S promoter. The CaMV35S and FMV35S promoters are active in a variety of transformed plant tissues and most plant organs (e.g., callus, leaf, seed and root). Enhanced or duplicate versions of the CaMV35S and FMV35S promoters are particularly useful in the practice of this invention (U.S. Pat. No. 5,378,619, incorporated herein by reference in its entirety). Other useful nopaline synthase (NOS) and octopine synthase (OCS) promoters (which are carried on tumor-inducing plasmids of A. tumefaciens), the cauliflower mosaic virus (CaMV) 19S promoters, a maize ubiquitin promoter, the rice Actl promoter and the Figwort Mosaic Virus (FMV) 35S promoter (see e.g., U.S. Pat. No. 5,463,175; incorporated herein by reference in its entirety). It is understood that this group of exemplary promoters is non-limiting and that one skilled in the art could employ other promoters that are not explicitly cited here in the practice of this invention.

Promoters that are active in certain plant tissues (i.e. tissue specific promoters) can also be used to drive expression of KP4. Expression of KP4 in the tissue that is typically infected by the fungal pathogens is anticipated to be particularly useful. Thus, expression in reproductive tissues, seeds, roots, or leaves can be particularly useful in combating infection of those tissues by certain fungal pathogens in certain crops. Examples of useful tissue-specific, developmentally regulated promoters include but are not limited to the β-conglycinin 7S promoter (Doyle et al., 1986), seed-specific promoters (Lam and Chua, 1991), and promoters associated with napin, phaseolin, zein, soybean trypsin inhibitor, ACP, stearoyl-ACP desaturase, or oleosin genes. Examples of root-specific promoters include but are not limited to the RB7 and RD2 promoters respectively described in U.S. Pat.Nos. 5,459,252 and 5,837,876.

Another class of useful promoters are promoters that are induced by various environmental stimuli. Promoters that are induced by environmental stimuli include but are not limited to promoters induced by heat (i.e. heat shock promoters such as Hsp70), promoters induced by light (i.e. the light-inducible promoter from the small subunit of ribulose 1,5-bisphosphate carboxylase, ssRUBISCO, a very abundant plant polypeptide), promoters induced by cold (i.e. COR promoters), promoters induced by oxidative stress (i.e. Catalase promoters), promoters induced by drought (i.e. the wheat Em and rice rab16A promoters) and promoters induced by multiple environmental signals (i.e. rd29A promoters, Glutathione-S-transferase (GST) promoters).

Promoters that are induced by fungal infections in plants can also be used to drive expression of KP4. Useful promoters induced by fungal infections include those promoters associated with genes involved in phenylpropanoid metabolism (e.g., phenylalanine ammonia lyase, chalcone synthase promoters), genes that modify plant cell walls (e.g., hydroxyproline-rich glycoprotein, glycine-rich protein, and peroxidase promoters), genes encoding enzymes that degrade fungal cell walls (e.g., chitinase or glucanase promoters), genes encoding thaumatin-like protein promoters, or genes encoding proteins of unknown function that display significant induction upon fungal infection. Maize and Flax promoters, designated as Mis1 and Fist, respectively, are also induced by fungal infections in plants and can be used (U.S. Patent Application 20020115849).

An intron may also be included in the DNA expression construct, especially in instances when the sequence of interest is to be expressed in monocot plants. For monocot plant use, introns such as the maize hsp70 intron (U.S. Pat. No. 5,424,412; incorporated by reference herein in its entirety), the maize ubiquitin intron, the Adh intron 1 (Callis et al., 1987), the sucrose synthase intron (Vasil et al., 1989) or the rice Act1 intron (McElroy et al., 1990) can be used. Dicot plant introns that are useful include introns such as the CAT-1 intron (Cazzonnelli and Velten,2003), the pKANNIBAL intron (Wesley et al., 2001; Collier et al., 2005), the PIV2 intron (Mankin et al., 1997) and the “Super Ubiquitin” intron (U.S. Patent No. 6,596,925, incorporated herein by reference in its entirety; Collier et al., 2005) that have been operably integrated into transgenes. It is understood that this group of exemplary introns is non-limiting and that one skilled in the art could employ other introns that are not explicitly cited here in the practice of this invention.

A variety of transcriptional terminators are available for use in the DNA constructs of the invention. These are responsible for the termination of transcription beyond the transgene and its correct polyadenylation. Appropriate transcriptional terminators are those that are known to function in the relevant plant system. Representative plant transcriptional terminators include the CaMV 35S terminator, the tml terminator, the nopaline synthase terminator (NOS ter), and the pea rbcS E9 terminator. In certain embodiments, the inventions utilize the oleosin terminator and/or napin terminator. With regard to RNA polymerase III terminators, these terminators typically comprise a—52 run of 5 or more consecutive thymidine residues. In one embodiment, an RNA polymerase III terminator comprises the sequence TTTTTTT. These can be used in both monocotyledons and dicotyledons.

For certain target species, different antibiotic or herbicide selection markers can be included in the DNA constructs of the invention. Selection markers used routinely in transformation include the npt II gene (Kan), which confers resistance to kanamycin and related antibiotics, the bar gene, which confers resistance to the herbicide phosphinothricin, the hph gene, which confers resistance to the antibiotic hygromycin, the dhfr gene, which confers resistance to methotrexate, and the EPSP synthase gene, which confers resistance to glyphosate (U.S. Pat. Nos. 4, 940,935 and 5,188,642).

Screenable markers may also be employed in the DNA constructs of the present invention, including for example the β-glucuronidase or uidA gene (the protein product is commonly referred to as GUS), isolated from E. coli, which encodes an enzyme for which various chromogenic substrates are known; an R-locus gene, which encodes a product that regulates the production of anthocyanin pigments (red color) in plant tissues; a β-lactamase gene, which encodes an enzyme for which various chromogenic substrates are known (e.g., PADAC, a chromogenic cephalosporin); a xylE gene, which encodes a catechol dioxygenase that can convert chromogenic catechols; an α-amylase gene; a tyrosinase gene which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene, which allows for bioluminescence detection; an aequorin gene, which may be employed in calcium-sensitive bioluminescence detection; or a gene encoding for green fluorescent protein (PCT Publication WO 97/41228).

The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which has the genotype r-g, b, P1. Alternatively, any genotype of maize can be utilized if the C1 and R alleles are introduced together.

In some aspects, screenable markers provide for visible light emission or fluorescence as a screenable phenotype. Suitable screenable markers contemplated for use in the present invention include firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for population screening for bioluminescence, such as on tissue culture plates, or even for whole plant screening.

Many naturally fluorescent proteins including red and green fluorescent proteins and mutants thereof, from jelly fish and coral are commercially available (for example from CLONTECH, Palo Alto, Calif.) and provide convenient visual identification of plant transformation.

As noted above, the sequence of interest may also be operably linked to a 3′ non-translated region containing a polyadenylation signal. This polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene 3′ un-translated regions contain polyadenylate signals and represent non-limiting examples of such 3′ untranslated regions that can be used in the practice of this invention. It is understood that this group of exemplary polyadenylation regions is non-limiting and that one skilled in the art could employ other polyadenylation regions that are not explicitly cited here in the practice of this invention.

The DNA constructs that comprise the plant expression cassettes described above are typically maintained in various vectors. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in bothE.coli and Agrobacterium as well as one or more “border” sequences positioned so as to permit integration of the expression cassette into the plant chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts.

Methods for Obtaining Transgenic Plants with resistance to fungus

Techniques for transforming a wide variety of plant species are well known and described in the technical and scientific literature. See, for example, Weising et al, (1988) Ann Rev. Genet., 22:421-477. As described herein, the DNA constructs of the present invention typically contain a marker gene which confers a selectable phenotype on the plant cells. For example, the marker may encode biocide resistance, particularly antibiotic resistance, such as resistance to kanamycin, G418, bleomycin, hygromycin, or herbicide resistance, such as resistance to chlorsulfuron or Basta. Such selective marker genes are useful in protocols for the production of transgenic plants.

DNA constructs can be introduced into the genome of the desired plant host by a variety of conventional techniques. For example, the DNA construct may be introduced directly into the DNA of the plant cell using techniques such as electroporation and microinjection of plant cell protoplasts. Alternatively, the DNA constructs can be introduced directly to plant tissue using biolistic methods, such as DNA micro-particle bombardment. In addition, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al, (1984) EMBO J., 3:2717-2722. Electroporation techniques are described in Fromm et al, (1985) Proc. Natl. Acad. Sci. USA, 82:5824. Biolistic transformation techniques are described in Klein et al, (1987) Nature 327:70-7. The full disclosures of all references cited are incorporated herein by reference.

A variation involves high velocity biolistic penetration by small particles with the nucleic acid either within the matrix of small beads or particles, or on the surface (Klein et al, (1987) Nature, 327:70-73,). Although typically only a single introduction of a new nucleic acid segment is required, this method particularly provides for multiple introductions.

Agrobacterium tumefaciens-meditated transformation techniques are well described in the scientific literature. See, for example Horsch et al, (1984) Science, 233:496-498, and Fraley et al, (1983) Proc. Natl. Acad. Sci. USA, 90:4803.

Any of the KP4 expression vectors can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005,10; 433(7026):629-33. It is further understood that the KP4 expression vector can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the expression vector into the chromosome of a plant cell, a plant tissue or a plant.

Methods of introducing plant minichromosomes comprising plant centromeres that provide for the maintenance of the recombinant minichromosome in a transgenic plant can also be used in practicing this invention (U.S. Pat. 6,972,197). In these embodiments of the invention, the transgenic plants harbor the minichromosomes as extrachromosomal elements that are not integrated into the chromosomes of the host plant.

Transgenic plants are typically obtained by linking the gene of interest (i.e., in this case a KP4 expression vectors) to a selectable marker gene, introducing the linked transgenes into a plant cell, a plant tissue or a plant by any one of the methods described above, and regenerating or otherwise recovering the transgenic plant under conditions requiring expression of said selectable marker gene for plant growth. The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

Transgenic plants can also be obtained by linking a gene of interest (i.e., in this case a KP4 expression vector) to a scoreable marker gene, introducing the linked transgenes into a plant cell by any one of the methods described above, and regenerating the transgenic plants from transformed plant cells that test positive for expression of the scoreable marker gene. The scoreable marker gene can be a gene encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein or a chloramphenicol acetyl transferase protein.

When the expression vector is introduced into a plant cell or plant tissue, the transformed cells or tissues are typically regenerated into whole plants by culturing these cells or tissues under conditions that promote the formation of a whole plant (i.e. the process of regenerating leaves, stems, roots, and, in certain plants, reproductive tissues). The development or regeneration of transgenic plants from either single plant protoplasts or various explants is well known in the art (Horsch, R. B. et al. 1985). This regeneration and growth process typically includes the steps of selection of transformed cells and culturing selected cells under conditions that will yield rooted plantlets. The resulting transgenic rooted shoots are thereafter planted in an appropriate plant growth medium such as soil. Alternatively, transgenes can also be introduced into isolated plant shoot meristems and plants regenerated without going through callus stage tissue culture (U.S. Pat. No. 7,002,058). When the transgene is introduced directly into a plant, or more specifically into the meristematic tissue of a plant, seed can be harvested from the plant and selected or scored for presence of the transgene. In the case of transgenic plant species that reproduce sexually, seeds can be collected from plants that have been “selfed” (self-pollinated) or out-crossed (i.e. used as a pollen donor or recipient) to establish and maintain the transgenic plant line. Transgenic plants that do not sexually reproduce can be vegetatively propagated to establish and maintain the transgenic plant line. As used here, transgenic plant line refers to transgenic plants derived from a transformation event where the transgene has inserted into one or more locations in the plant genome. In a related aspect, the present invention also encompasses a seed produced by the transformed plant, a progeny from such seed, and a seed produced by the progeny of the original transgenic plant, produced in accordance with the above process. Such progeny and seeds will have a KP4 protein-encoding transgene stably incorporated into their genome, and such progeny plants will inherit the traits afforded by the introduction of a stable transgene in Mendelian fashion. All such transgenic plants having incorporated into their genome transgenic DNA segments encoding one or more KP4 proteins or polypeptides are aspects of this invention. It is further recognized that transgenic plants containing the DNA constructs described herein, and materials derived therefrom, may be identified through use of PCR or other methods that can specifically detect the sequences in the DNA constructs.

Once a transgenic plant is regenerated or recovered, a variety of methods can be used to identify or obtain a transgenic plant that expresses a plant pathogenic fungus inhibitory amount of KP4. One general set of methods is to perform assays that measure the amount of KP4 that is produced. For example, various antibody-based detection methods employing antibodies that recognize KP4 can be used to quantitate the amount of KP4 produced. Examples of such antibody based assays include but are not limited to ELISAs, RIAs, or other methods wherein a KP4-recognizing antibody is detectably labelled with an enzyme, an isotope, a fluorophore, a lanthanide, and the like. By using purified or isolated KP4 protein as a reference standard in such assays (i.e. providing known amounts of KP4), the amount of KP4 present in the plant tissue in a mole per gram of plant material or mass per gram of plant material can be determined. The KP4 protein will typically be expressed in the transgenic plant at the level of “parts per million” or “PPM” where microgram levels of KP4 protein are present in gram amounts of fresh weight plant tissue. In this case, 1 microgram of KP4 protein per 1 gram of fresh weight plant tissue would represent a KP4 concentration of 1 PPM. A plant pathogenic fungus inhibitory amount of KP4 protein is at least 0.05 PPM (i.e. 0.05 lug KP4 protein per gram fresh weight plant tissue). In preferred embodiments, a plant pathogenic fungus inhibitory amount of KP4 protein is at least 0.5 PPM. In more preferred embodiments, the amount of KP4 is at least 1.0 PPM. In the most preferred embodiments, the amount of KP4 protein is at least 2.0 PPM.

Alternatively, the amount of KP4-encoding mRNA produced by the transgenic plant can be determined to identify plants that express plant pathogenic fungus inhibitory amounts of KP4 protein. Techniques for relating the amount of protein produced to the amount of RNA produced are well known to those skilled in the art and include methods such as constructing a standard curve that relates specific RNA levels (i.e. KP4 mRNA) to levels of the KP4 protein (determined by immunologic or other methods). Methods of quantitating KP4 mRNA typically involve specific hybridization of a polynucleotide to either the KP4 mRNA or to a cDNA (complementary DNA) or PCR product derived from the KP4 RNA. Such polynucleotide probes can be derived from either the sense and/or antisense strand nucleotide sequences of the KP4 protein-encoding transgene. Hybridization of a polynucleotide probe to the KP4 mRNA or cDNA can be detected by methods including, but not limited to, use of probes labelled with an isotope, a fluorophore, a lanthanide, or a hapten such as biotin or digoxigenin. Hybridization of the labelled probe may be detected when the KP4 RNA is in solution or immobilized on a solid support such as a membrane. When quantitating KP4 RNA by use of a quantitative reverse-transcriptase Polymerase Chain Reaction (qRT-PCR), the KP4-derived PCR product can be detected by use of any of the aforementioned labelled polynucleotide probes, by use of an intercalating dye such as ethidium bromide or SYBR green, or use of a hybridization probe containing a fluorophore and a quencher such that emission from the fluorophore is only detected when the fluorophore is released by the 5′ nuclease activity of the polymerase used in the PCR reaction (i.e. a TaqMan™ reaction; Applied Biosystems, Foster City, Calif.) or when the fluorophore and quencher are displaced by polymerase mediated synthesis of the complementary strand (i.e. Scorpion™ or Molecular Beacon™ probes).Various methods for conducting qRT-PCR analysis to quantitate mRNA levels are well characterized (Bustin, S.A.; 2002). Fluorescent probes that are activated by the action of enzymes that recognize mismatched nucleic acid complexes (i.e. INVADER™, Third Wave Technologies, Madison, Wis.) can also be used to quantitate RNA. Those skilled in the art will also understand that RNA quantitation techniques such as Quantitative Nucleic Acid Sequence Based Amplification (Q-NASBA) can be used to quantitate KP4 protein-encoding mRNA and identify expressing plants.

Transgenic plants that express plant pathogenic fungus inhibitory amounts of KP4 can also be identified by directly assaying such plants for inhibition of the growth of a plant pathogenic fungus. Such assays can be used either independently or in conjunction with KP4-expression assays to identify the resistant transgenic plants. Infection of certain plants with certain plant pathogen fungi can result in distinctive effects on plant growth that are readily observed. Consequently, one can distinguish KP4-expressing transgenic plants by simply challenging such plants transformed with KP4-encoding transgenes with pathogenic plant fungi and observing reduction of the symptoms normally associated with such infections. Such observations are facilitated by co-infecting control plants that do not contain a KP4 encoding transgene with the same type and dose of plant pathogenic fungi used to infect the transgenic plants that contain a KP4-encoding transgene. Identification of transgenic plants that control or combat fungal infection can be based on observation of decreased disease symptoms, measurement of the decreased fungal growth in the infected plant (i.e., by determining the numbers of colony forming units per gram of infected tissue) and/or by measurement of the amount of mycotoxins present in infected plant tissue. The use of fungal disease severity assays and colony formation assays in conjunction with expression assays to identify transgenic MsDef1-expressing potato plants that are resistant to Verticillium dahliae has been described (U.S. Pat. No. 6,916,970 and Gao et al, 2000). It is similarly anticipated that a variety of KP4-expressing transgenic plants that combat or control fungal pathogens can be identified by scoring transgenic plants for resistance to fungal pathogens that infect those plants. Examples of KP4transgene- conferred fungal resistance that can be assayed by observing reductions in disease symptoms or reductions in fungal growth include, but are not limited to, resistance of transgenic corn to Fusarium verticillioides, Fusarium moniliforme, Stenocarpella maydis, and/or Cercospora zeae-maydis; resistance of transgenic wheat to head blight (Fusarium graminearum), powdery mildew (Erysiphe graminis f. sp. tritici), leaf rust (Puccinia recondita f. sp. tritici), or stem rust (P. gramimis f. sp. tritici); resistance of transgenic cotton to Fusarium oxysporum; resistance of transgenic rice to Magnaporthe grisea and Rhizoctonia solani, resistance of transgenic soybean to Asian Soybean rust (Phakopsora pachyrhizi), Phytophthora Root Rot (Phytophthora sp.), White Mold (Sclerotinia sp.), Sudden Death Syndrome (Fusarium solani) and/or Brown Stem Rot (Phialophora gregata), and resistance of transgenic banana to Fusarium wilt disease (F. oxysporum f. sp. cubense).

Transgenic plants that express plant pathogenic fungus inhibitory amounts of KP4 can also be identified by measuring decreases in the adverse effects cause by fungal growth in a plant. Such decreases can be ascertained by comparing the extent of the adverse effect in a KP4 expressing transgenic plant relative to a control plant that does not express KP4. Adverse effects of fungal growth in a plant that can be measured include any type of plant tissue damage or necrosis, any type of plant yield reduction, any reduction in the value of the crop plant product, and/or production of undesirable fungal metabolites or fungal growth by-products including but not limited to mycotoxins. Mycotoxins comprise a number of toxic molecules produced by fungal species, including but not limited to polyketides (including aflatoxins, demethylsterigmatocystin, O-methylsterigmatocystin etc.), fumonisins, alperisins (e.g., A₁, A₂, B₁, B₂), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown. Mass., USA).

A wide variety of plants can be transformed with KP4 expressing vectors to obtain transgenic plants that combat or control fungal infections. Transgenic monocot plants obtainable by the expression vectors and methods described herein include but are not limited to barley, corn, flax, oat, rice, rye, sorghum, turf grass, sugarcane, and wheat. Transgenic dicot plants obtainable by the expression vectors and methods described herein include but are not limited to alfalfa, Arabidopsis, barrel medic, banana, broccoli, bean, cabbage, canola, carrot, cassava, cauliflower, celery, citrus, cotton, cucurbits, eucalyptus, garlic, grape, onion, lettuce, pea, peanut, pepper, potato, poplar, pine, sunflower, safflower, soybean, strawberry, sugar beet, sweet potato, tobacco, and tomato.

Other proteins conferring certain advantages may likewise be co-expressed with the DNAs encoding the polypeptides of the present invention; including: (1) DNAs encoding enzymes such as glucose oxidase (which converts glucose to gluconic acid, concomitantly producing hydrogen peroxide which confers broad spectrum resistance to plant pathogens); pyruvate oxidase; oxalate oxidase; cholesterol oxidase; amino acid oxidases; and other oxidases that use molecular oxygen as a primary or secondary substrates to produce peroxides, including hydrogen peroxide; (2) pathogenesis-related proteins such as SAR8.2a and SAR8.2b proteins; the acidic and basic forms of tobacco PR-la, PR-lb, PR-lc, PR-1′, PR-2, PR-3, PR-4, PR-5, PR-N, PR-O, PR-0′, PR-P, PR-Q, PR-S, and PR-R proteins; chitinases such as tobacco basic chitinase and cucumber chitinase/lysozyme; peroxidases such as cucumber basic peroxidase; glucanases such as tobacco basic glucanase; osmotin-like proteins; (3) viral capsid proteins and replicases of plant viruses; (4) plant R-genes (resistance genes) and homologs thereof, including but not limited to Arabidopsis RPS2 (Bent et al., 1994), Arabidopsis RPM1 (Grant et al., 1995), tobacco N-gene and N′-gene, tomato Cf-9, flax L6, and rice Xa21; (5) pathogen Avr genes, such as Cladosporium fulvum Avr9, that can be expressed using pathogen- or chemical-inducible promoters; (6) genes that are involved in the biosynthesis of salicylic acid, such as benzoic acid 2-hydroxylase; and (7) defensin proteins including but not limited to, MsDef1, MtDef2, MtDef4, Rs-AFP1 and Rs-AFP2. Various MtDef4 proteins with antifungal activity that can be used are disclosed in US Patent Application Publication No. 20080201800.

### Transgenic Plants

In certain embodiments, the invention contemplates a transgenic plant comprising within its genome:
a nucleotide sequence encoding a fusion protein comprising a mature KP4 protein that is operatively linked a heterologous signal peptide that is operably linked to a first set of expression control sequences that drive expression of the fusion protein in the plant cell;
wherein the KP4 protein is expressed primarily in the plant cell apoplast and provides for at least 50% inhibition of a plant pathogenic fungal infection relative to a control plant that lacks said recombinant nucleic acid construct.

In different embodiments, the transgenic organisms contain one or more of the DNA constructs and expression vectors of the invention as defined herein as a part of the organism, the DNA constructs having been introduced by transformation of the organism. In certain embodiments, the KP4 protein comprises an amino acid sequence selected from Table Dl. In some embodiments the KP4 gene is codon optimized. In certain embodiments the KP4 is codon optimized for high level expression in a monocot. In certain embodiments the KP4 is codon optimized for high level expression in a dicot,

In some aspects the nucleic acid encoding the codon optimized mature KP4 is at least 80% identical to SEQ. ID. No. 3. In one aspect the KP4 protein encoded by the nucleic acid has an amino acid sequence which is at least about 80% identical to SEQ. ID. No. 4.

In some embodiments the heterologous signal peptide is codon optimized for expression in the plant of interest. In some aspects the signal peptide is from MsDef1. In some aspects the nucleic acid encoding the codon optimized MsDef1 signal peptide is at least 80% identical to SEQ. ID. No. 5. In one aspect the nucleic acid encoding the signal peptide encodes an amino acid sequence which is at least about 80% identical to SEQ. ID. No. 6.

In another aspect such transgenic organisms are characterized by having a KP4 content of about of at least about, 0.5 PPM, about 1 PPM, about 1.5 PPM, or about 2 PPM. Where 1 microgram of KP4 protein per 1 gram of fresh weight plant tissue represents a KP4 concentration of 1 PPM.

In certain embodiments of the transgenic plants, the mature KP4 protein is expressed primarily in the apoplastic space within the tissue of the transgenic plant. In this context, the term “primarily” means that the relative expression of the mature KP4 protein is at least about 150%, or at least about 200%, or at least about 300%, or at least about 400%, or at least about 500% higher in the apoplastic space (on a dry weight by dry weight basis) compared to any other plant tissue, in the mature full developed plant, when grown under standard growth conditions.

In certain embodiments the transgenic plant provides for at least 75%, at least 85%, or at least 95% inhibition of a plant pathogenic fungal infection relative to a control plant that lacks said recombinant nucleic acid construct.

In certain embodiments the transgenic plant provides for at least 50% inhibition of a biotrophic plant pathogenic fungus and/or at least 50% inhibition of a necrotrophic plant pathogenic fungus. In some aspects, the biotrophic plant pathogenic fungus is selected from the group consisting of Ustilago species, Podosphaera species, Erysiphe species, Phakopsora species, and Puccinia species. In some aspects, the necrotrophic plant pathogenic fungus is selected from the group consisting of Alternaria species, Botrytis species, Colletotrichum species, Cercospora species, Fusarium species, Phoma species, Phytophthora species, Pythium species, Sclerotinia species, and Verticillium species.

In certain embodiments the transgenic plant is a monocot plant is selected from the group consisting of barley, corn, maize, flax, oat, rice, rye, sorghum, turf grass, sugarcane, and wheat. In one aspect, the transgenic plant is corn. In one aspect, the transgenic plant is maize.

In certain embodiments the transgenic plant is a dicot plant is selected from the group consisting of alfalfa, Arabidopsis, barrel medic, banana, broccoli, bean, cabbage, canola, carrot, cassava, cauliflower, celery, citrus, cotton, a cucurbit, eucalyptus, garlic, grape, onion, lettuce, pea, peanut, pepper, potato, poplar, pine, sunflower, safflower, soybean, strawberry, sugar beet, sweet potato, tobacco, and tomato.

In certain embodiments the transgenic plant further comprises a second recombinant nucleic acid construct that provides for expression of MsDef1, MtDef2, MtDef4, Rs-AFP1, or Rs-AFP2.

In any of these transgenic characteristics, it will be understood that the transgenic organism will be grown using standard growth conditions as disclosed in the Examples, and compared to the equivalent wild type species.

### EXAMPLES

### Example 1

### Construction of a KP4 Expression Vector and Transformation of Maize

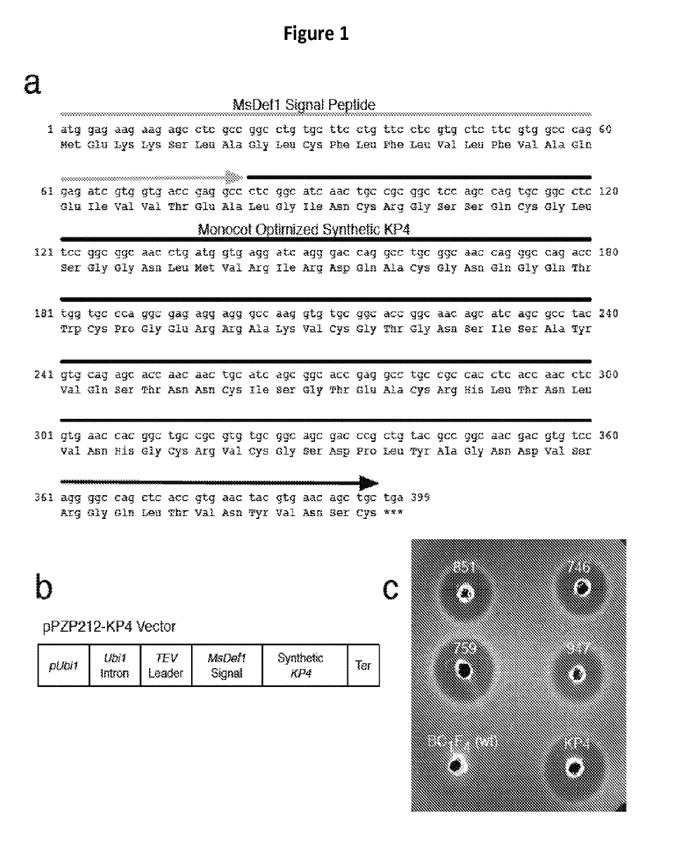

For expression in transgenic maize, a chimeric KP4 gene was designed in which the nucleotide sequence encoding 105-amino acid KP4 was fused to the carboxy-terminus of the 27-amino acid signal peptide sequence of a plant defensin, MsDef1 (FIG. 1A). A monocot codon-optimized chimeric MsDef1 signal peptide-KP4 gene was synthesized (GenScript Corporation, N.J.). This chimeric KP4 gene expression cassette consisting of the maize ubiquitin (UbilA) promoter and intron, the tobacco etch virus leader, the monocot codon-optimized MsDef1 signal peptide/ mature KP4 coding sequence and the CaMV 35S terminator was cloned between the T-DNA borders into the binary vector pZP212 (Hajdukiewicz et al., 1994) as shown in FIGS. 1B and 4A. The resulting construct was introduced into A. tumefaciens strain EHA101 for maize transformation.

Maize inbred line H99 was transformed using previously described protocols (Sidorov, 2005) with the modification that immature embryos instead of mature seeds were used as starting material. The primary transgenic maize plants (T₀)) were out-crossed to non-transgenic public inbred line B73 to generate BC₀F₁ seeds. Based on the Mendelian segregation of 1:1 indicative of a single-insert, the BC₀F₁ plants were self-pollinated twice to generate BC₀F₃ lines used subsequently for corn smut resistance evaluations. All plants were grown using the standard greenhouse growth conditions used for maize.

Transgenic corn plant lines that expressed the KP4 protein were identified using standard PCR methods and ELISA assays. Antibodies were raised against purified KP4 by Sigma Genosys (The Woodlands, Tex., USA) and conjugated to horseradish peroxidase using the SurLINK™ HRP Conjugation Kit (KPL, Gaithersburg, Md.). Using ELISA assays standardized with purified KP4 (Gu, 1995), the resistant homozygous lines expressed 4-7ppm of KP4. However, since there are five disulfide bonds in KP4, there were concerns as to whether the protein would be folded properly during cellular export. To test for this, fresh leaf material was ground in PBS and tested for antifungal activity. As shown in FIG. 1C, KP4 expressed in the transgenic lines is quite effective at killing sensitive strains of U. maydis, demonstrating that the transgenic protein is properly folded and active.

### Example 2

### Inhibition of Fungal Infections in Transgenic Maize Expressing KP4

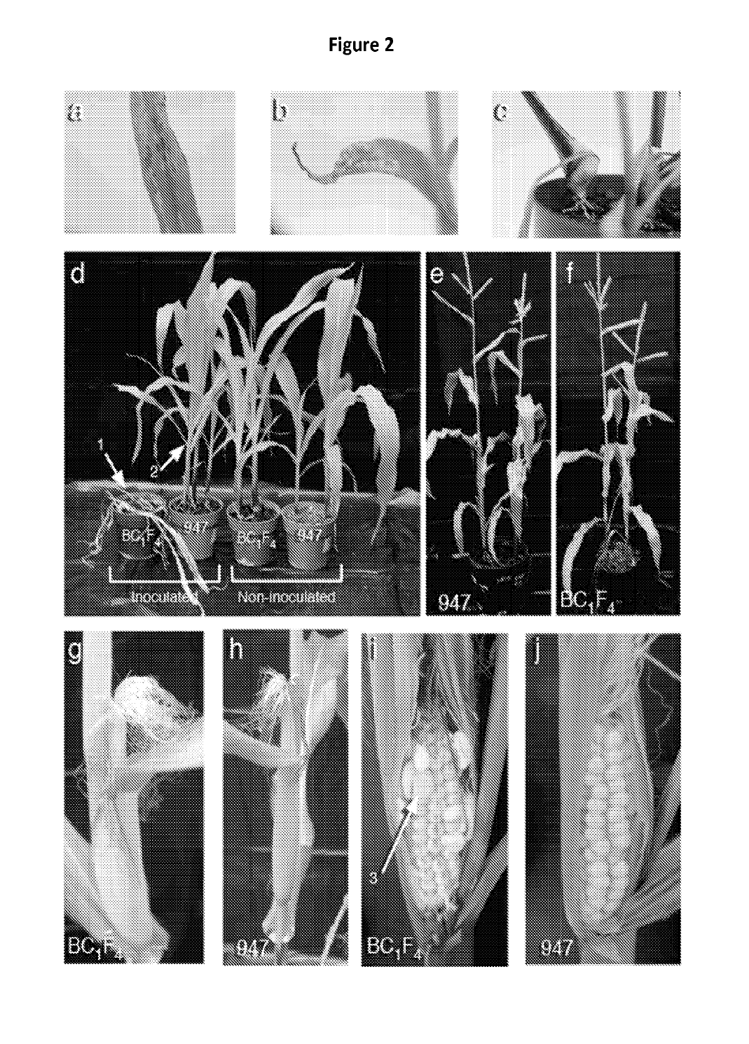

With evidence that the transgenic lines of maize were producing bioactive KP4 protein, it was necessary to ascertain the ability of this antifungal protein to protect the plants against fungal challenges. U. maydis KP4 sensitive wild-type strains 1/2 (albl) and 2/9 (a2b2, near isogenic to 1/2) were grown in potato dextrose broth (PDB) to an OD600 of 1.0 (˜1×10⁷ cells/mL). Cells were centrifuged and resuspended in water to 1×106 cells/mL. These strains were mixed in equal amounts immediately prior to inoculation. Corn plants were established in a fine grade composted pine bark mixed with vermiculite in a 3:1 ratio in 9 cm plastic pots under 16 h light at 28° C. and 8 h dark at 20° C. in a CONVIRON™ growth chamber. Using a hypodermic needle and syringe, 3.0 mL of the cell suspension was injected into the stems of 7-day-old maize plants. Initial disease symptoms were observed 7-10 days post-inoculation (dpi). Disease symptoms were recorded at 7, 10, 14 and 21 dpi using the method of Gold et al.14. Surviving plants were transplanted into 24 cm plastic pots and grown under the same conditions as mentioned above for 80-90 days. Upon the appearance of silks, maize ears were inoculated with 3.0 mL of the fungal cell suspension. Ear symptoms were observed 14 dpi. All data was subjected to analysis of variance (ANOVA) and Duncan's Multiple Range Test (DMRT) via DSAASTAT statistical software, Version 1.019215. U. maydis KP4 sensitive wild-type strains 1/2 (alb]) and 2/9 (a2b2, near isogenic to 1/2) were grown in potato dextrose broth (PDB) to an OD₆₀₀ of 1.0 (˜1×10⁷ cells/ml). Cells were centrifuged and resuspended in water to a density of 1×10⁶ cells/ml and mixed in equal amounts immediately prior to inoculation. Using a hypodermic needle and syringe, 3.0 mL of the cell suspension was injected into the stems of 7-day-old maize plants. In wild type plants, initial disease symptoms were observed 7-10 days post-inoculation (dpi; FIG. 2A-D). Disease symptoms were recorded at 7, 10, 14 and 21 dpi using the method of Gold et al. (Gold, 1997). Surviving plants were transplanted into 24 cm plastic pots for 80-90 days. Upon the appearance of silks, maize ears were inoculated with an additional 3.0 mL of the fungal cell suspension and ear symptoms were observed 14 dpi. All data was subjected to analysis of variance (ANOVA) and Duncan's Multiple Range Test (DMRT) via DSAASTAT statistical software, Version 1.0192 {Onofri, 2006 #39401.

Ten independently generated KP4 transgenic maize lines (i.e. distinct transgenic events) were planted and developed normally when compared to the control line H199/B73 (BC₁F₄; FIG. 2E,F). Seven day old seedlings were inoculated with a mixture of the wild-type U. maydis KP4 sensitive strains 1/2 and 2/9. Disease symptoms were observed and scored at 10 dpi. To determine whether plants resist U. maydis infection or simply delay U. maydis infection, KP4 transgenic lines plants were grown to 21 dpi. Disease symptoms were absent 21 dpi in several KP4 transgenic plants, while the wild type maize line (BC1F4) exhibited plant death (FIG. 2D). Five KP4 transgenic lines, 851, 947, 1040, 885, and 8510 showed strong resistance toU. maydis infection (Table 1). Four KP4 transgenic lines, 746, 759, 810, and 923 showed incomplete resistance to U. maydis infection. One KP4 transgenic line, 826, showed no resistance to U. maydis infection. Transgenic plants were then transplanted into large pots and observed for any developmental differences in comparison to the wild type. Three-month-old transgenic plants developed normally when compared with wild type plants (FIG. 2E,F). Secondary inoculation with 1/2 and 2/9 strains directly into maize ears was applied upon the appearance of silks (FIG. 2G, H). Two weeks later, plant tumors or galls were observed in the ears of the wild-type maize line (BC1F4) and transgenic 826 (FIG. 2I), while the ears of the KP4 transgenic lines 851, 746, 759, 947, 1040, 810, 885, 923 and 8510 were healthy (FIG. 2F).

Antifungal resistance of the various transgenic lines are consistent with the ELISA assays on the leaf material. Lines 759, 810, and 923 all showed partial resistance and, in fact, are still segregating and not yet homozygous lines. As shown in Table 1, line 826 was completely susceptible to infection and was a null segregant that did express KP4. The five highly resistant lines were all homozygous and expressed high levels of KP4. The only transgenic event where resistance did not correlate well with expression was line 746. In this case, the ELISA results showed good expression of KP4, but the plants only had partial resistance to infection. The reasons for this are not clear, but it may be due to a lower level of KP4 expression that was not evident in the highly sensitive ELISA assay. Nevertheless, there is a strong correlation between KP4 expression and high resistance to Ustilago maydis infection. Importantly, these results demonstrate that KP4, expressed throughout the plant, can prevent infection in all organs while recent results suggest that naturally occurring immunity is encode by a number of genes in an organ-dependent manner (Baumgarten, 2007).

TABLE E1
Disease symptoms caused by U. maydis
infection on KP4 transgenic maize.

| Events | | | | | | | | |
|---|---|---|---|---|---|---|---|---|
| (Maize | Total | Disease Score | | | | | | Disease |
| Lines) | # Plants | 0 | 1 | 2 | 3 | 4 | 5 | Index |
| 947 | 53 | 49 | 4 | 0 | 0 | 0 | 0 | 0.08^{b} |
| 1040 | 59 | 54 | 5 | 0 | 0 | 0 | 0 | 0.08^{b} |
| 972 | 40 | 34 | 6 | 0 | 0 | 0 | 0 | 0.15^{b} |
| 885 | 40 | 32 | 8 | 0 | 0 | 0 | 0 | 0.20^{b} |
| 851 | 60 | 47 | 12 | 1 | 0 | 0 | 0 | 0.23^{b} |
| 810 | 40 | 13 | 16 | 7 | 4 | 0 | 0 | 1.05^{ab} |
| 923 | 40 | 15 | 12 | 8 | 5 | 0 | 0 | 1.08^{ab} |
| 746 | 60 | 18 | 24 | 12 | 6 | 0 | 0 | 1.10^{ab} |
| 759 | 58 | 9 | 20 | 19 | 7 | 3 | 0 | 1.57^{ab} |
| 826 (null) | 40 | 2 | 5 | 3 | 12 | 17 | 1 | 3.00^{a} |
| H99/B73 (wt) | 60 | 2 | 11 | 10 | 22 | 13 | 2 | 2.65^{a} |
| Non- | 18 | 15 | 3 | 0 | 0 | 0 | 0 | 0.17^{b} |
| infected 947 | | | | | | | | |
| Non- | 20 | 18 | 2 | 0 | 0 | 0 | 0 | 0.10^{b} |
| infected wt | | | | | | | | |

Results from three pathogenicity experiments. The disease ratings are as follows: 0=no symptoms; 1=anthocyanin and/or chlorosis; 2=small leaf galls; 3=small stem galls; 4=basal galls; 5=plant death. 851, 746, 759, 947, 1040, 810, 826, 885, 923, and 8510 are ten independently generated KP4 transgenic maize lines. H99/B73 is the wild-type (wt) maize line from which KP4 transgenic plants were generated. Symptoms were scored 10 dpi. Values with different superscripts in the disease index column are significantly different (p<0.05) in Duncan's Multiple Range Test.

### Example 3

### Expression of Bioactive KP4 in Transgenic Soybean Plants

For soybean transformation, a chimeric gene encoding a heterologous MsDef1 signal peptide that is operably linked to a synthetic KP4 gene encoding a mature KP4 protein (SEQ ID NO: 1) was placed under the control of the Figwort mosaic virus 35S promoter that is known to give high level expression of transgenes in soybean (vector EHA105)(FIG. 4B). The 5′FMV 35S promoter/MsDef1 signal peptide/KP4 gene/nopaline synthase 3′ chimeric gene construct thus generated was introduced into soybean (Glycine max L. Merrill cv Jack) using Agrobacterium tumefaciens-mediated transformation and regeneration (Zhang, 1999;Clemente, 2000).

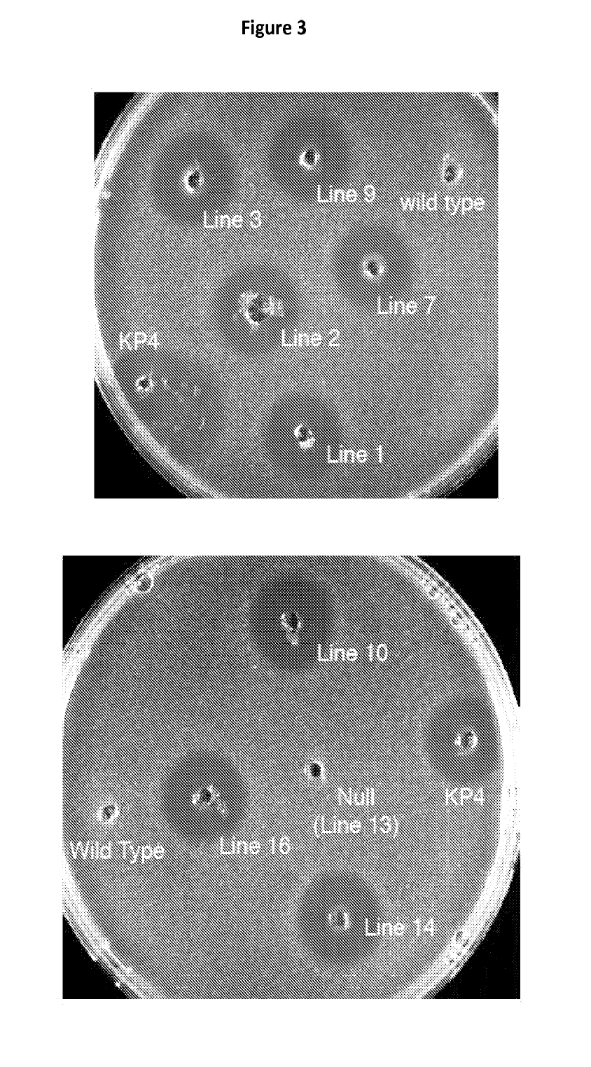

Nine independent transgenic soybean lines were generated that were BASTA resistant. To test for production of bioactive KP4, 50mg of leaf tissue was ground in lml of phosphate buffer and 20 μl of this solution was added to wells in agar suspended Ustilago maydis (P2 strain). Active KP4 causes a clearing zone around the well. As shown in FIG. 3A and 3B, all but one soybean line (Null (line 13) in FIG. 3B) produced significant amounts of active KP4. From this killing activity, the transgenic soybean lines are estimated to produce on the order of 1-5 ppm KP4 in the leaf tissue.

The disclosed embodiments are merely representative of the invention, which may be embodied in various forms.

### Non-Patent Publications

Baumgarten, A. M., J. Suresh, G. May, and R. L. Phillips, Mapping QTLs contributing to Ustilago maydis resistance in specific plant tissues of maize. Theor. Appl. Genet., 2007. 114: p. 1229-1238.

Broothaerts W, Mitchell HJ, Weir B, Kaines S, Smith L M, Yang W, Mayer J E,Roa-Rodriguez C, Jefferson RA. 2005. Gene transfer to plants by diverse species of bacteria. 2005. Nature. 433(7026):629-33.

Bustin, S. A. Quantification of mRNA using real-time reverse transcription PCR (RT-PCR): trends and problems. Journal of Molecular Endocrinology (2002) 29, 23-39.

Bussey, H., Effects of yeast killer factor on sensitive cells. Nature New Biology, 1972. 235: p. 73-75.

Callis, J, Fromm, M, Walbot, V. (1987) Introns increase gene expression in cultured maize cells. Genes Dev. 1987 Dec;1(10):1183-200.

Cazzonnelli, C. I. and J. Velten. (2003) Construction and Testing of an Intron-Containing Luciferase Reporter Gene From Renilla reniformis. Plant Molecular Biology Reporter 21: 271-280.\

Clemente, T. E., B. J. LaVallee, A.R. Howe, D. Conner-Ward, R. J. Rozman, P.E. Hunter, D. L. Broyles, D. S. Kasten, and M. A. Hinchee, Progeny Analysis of Glyphosate Selected Transgenic Soybeans Derived from Agrobacterium-Mediated Transformation. Crop Sci., 2000. 40: p. 797-803.

Frame, B.R., Shou, H., Chikwamba, R. K., Zhang, Z., Xiang C., Fonger,T. M., Pegg S E, Li B, Nettleton D S, Pei D, and Wang K. (2002) Agrobacterium tumefaciens-mediated transformation of maize embryos using a standard binary vector system. Plant Physiol. 129(1):13-22.

Gage, M. J., J. Bruenn, M. Fischer, D. Sanders, and T. J. Smith, KP4 fungal toxin inhibits growth in Ustilago maydis by blocking calcium uptake. Mol Microbiol, 2001. 41(4): p. 775-85.

Ganesa, C., W. H. Flurkey, Z. I. Randhawa, and R. F. Bozarth, Ustilago maydis virus P4 killer toxin: Characterization, partial amino terminus sequence, and evidence for glycosylation. Arch. Biochem. Biophysics, 1991. 286: p. 195-200.

Ganesa, C., Y.-H. Chang, W. H. Flurkey, and R. F. Bozarth, Purification and molecular properties of the toxin coded by Ustilago maydis virus P4. Biophys. Biochem. Res. Commun , 1989. 162(2): p. 651-6

Gao, A., Hakimi, S. M., Mittanck, C. A., Wu, Y., Woerner, M. B., Stark, D. M., Shah, D. M., Liang, J., and Rommens, C. M. T. (2000). Fungal pathogen protection in potato by expression of a plant defensin peptide. Nature Biotechnology 18, 1307-1310.

Gold, S. E., S. M. Brogdon, M. E. Mayroga, and J. W. Kronstad, The Ustilago maydis regulatory subunit of a cAMP-dependent protein kinase is required for gall formation in maize. Plant Cell, 1997. 9: p. 1585-1594.

Gu, F., A. Khimani, S. G. Rane, W.H. Flurkey, R. F. Bozarth, and T. J. Smith, Structure and function of a virally encoded fungal toxin from Ustilago maydis: a fungal and mammalian Ca2+channel inhibitor. Structure, 1995. 3(8): p. 805-14.

Hankin, L. and J. E. Puhalla, Nature of a factor causing interstrain lethality in Ustilago maydis. Phytopathology, 1971. 61: p. 50-53.

Hanks, J. N., Snyder, A. K., Graham, M. A., Shah, R. K., Blaylock, L. A., Harrison, M. J., and Shah, D. M. (2005). Defensin gene family in Medicago truncatula: structure, expression and induction by signal molecules. Plant Mol Biol 58, 385-399.

Horsch, R. B., J. E. Fry, N. Hoffman et al. (1985) A simple and general method for transferring genes into plants. Science. 227: 1229-1231.

Koehler S M, and Ho, T H. (1990) Hormonal regulation, processing, and secretion of cysteine proteinases in barley aleurone layers. Plant Cell. (8):769-83.

Lam E, and Chua N H. (1991) Tetramer of a 21-base pair synthetic element confers seed expression and transcriptional enhancement in response to water stress and abscisic acid. J Biol Chem. 1991 Sep. 15; 266(26):17131-5.

McElroy, D, Zhang W, Cao J, Wu R. 1990. Isolation of an efficient actin promoter for use in rice transformation. The Plant Cell, Vol. 2, 163-171

Onofri, A., Enhancing Excel capability to perform statistical analyses in agriculture applied research. Computational Statistics and data analysis, in Statistical Software Newsletters. 2006, International Association for statistical Computing.

Park, C. M., J.A. Bruenn, C. Ganesa, W. F. Flurkey, R.F. Bozarth, and Y. Koltin, Structure and heterologous expression of Ustilago maydis viral toxin KP4. Mol. Microbiol., 1994. 11(1): p. 155-164.

Rispail, N., D. M. Soanes, C. Ant, R. Czajkowski, A. Grünler, R. Huguet, Perez-Nadales, P. E., A., E. Sartorel, V. Valiante, M. Yang, R. Beffa, A. A. Brakhage, N. A. R. Gow, R. Kahmann, M. H. Lebrun, H. Lenasi, J. Perez-Martin, N. J. Talbot, J. Wendland, and A. Di Pietro, Comparative genomics of MAP kinase and calcium—calcineurin signalling components in plant and human pathogenic fungi. Fungal Genetics and Biology, 2009. 46: p. 287-298.

Sambrook, J., and Russell, D. W. (2001). Molecular Cloning: A Laboratory Manual. (Cold Sprong Harbor Laboratory Press, Cold Sprong Harbor, New York).

Schlaich, T., B. Urbaniak. N. Malgrass, E. Ehler, C. Buffer, L. Meier, and C. Sautter, Field resistance to Tilletia caries provided by a specific antifungal virus gene in genetically engineered wheat. Plant Biotechnol J., 2006. 4: p. 63-75.

Sidorov, V, Gilbertson, L, Addae, P, and Duncan, D. (2006) Agrobacterium-mediated transformation of seedling-derived maize callus. Plant Cell Rep. 2006 Apr;25(4):320-8. (Epub 2005 Oct 27)

Sidorov, V., L. Gilbertson, P. Addae, and D. Duncan, Agrobacterium-mediated transformation of seedling-derived maize callus. Plant Cell Rep, 2005. 25: p. 320-328.

Vasil V, Clancy M, Ferl R J, Vasil I K, Hannah L C. (1989) Increased Gene Expression by the First Intron of Maize Shrunken-1 Locus in Grass Species. Plant Physiol. 1989 Dec;91(4):1575-1579.

Widmer, F., Assessing Effects of Transgenic Crops on Soil Microbial Communities. Adv Bichem Engin/Biotechnol 2007. 107: p. 207-234.

Voth, P. D., L. Mairura, B. E. Lockhart, and G. May, Phylogeography of Usilago maydis virus III in the USA and Mexico. J. Gen. Virol, 2006. 87: p. 3433-3441.

Young, T., Killer Yeasts, in The Yeasts, A. H. Rose and J.S. Harrison, Editors. 1987, Academic Press: Orlando, Fla. p. 131-164.

Zhang, Z., A. Xing, P. Staswick, and T. E. Clemente, The use of glufosinate as a selective agent in Agrobacterium-mediated transformation of soybean. Plant Cell, Tiss. Organ Cult., 1999. 56: p. 37-46.

What is claimed is:

1. A transgenic plant comprising a recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence, wherein said transgenic plant expresses said KP4 protein in the apoplast and provides for at least 50% inhibition of a plant pathogenic fungal infection relative to a control plant that lacks said recombinant nucleic acid construct.

2. The transgenic plant of claim 1, wherein said transgenic plant provides for at least 75%, at least 85%, or at least 95% inhibition of a plant pathogenic fungal infection relative to a control plant that lacks said recombinant nucleic acid construct.

3. The transgenic plant of claim 1, wherein said transgenic plant provides for at least 50% inhibition of a biotrophic plant pathogenic fungus and/or at least 50% inhibition of a necrotrophic plant pathogenic fungus.

4. The transgenic plant of claim 3, wherein said biotrophic plant pathogenic fungus is selected from the group consisting of Ustilago species, Podosphaera species, Erysiphe species, Phakopsora species, and Puccinia species.

5. The transgenic plant of claim 3, wherein said necrotrophic plant pathogenic fungus is selected from the group consisting of Alternaria species, Botrytis species, Colletotrichum species, Cercospora species, Fusarium species, Phoma species, Phytophthora species, Pythium species, Sclerotinia species, and Verticillium species.

6. The transgenic plant of claim 3, wherein said transgenic plant is a monocot plant or a dicot plant and wherein said non-native nucleic acid sequence comprises one or more non-native codons that are more abundant in monocot plant genes and/or one or more non-native codons that are more abundant in dicot plant genes.

7. The transgenic plant of claim 1, wherein said plant is a monocot plant is selected from the group consisting of barley, corn, flax, oat, rice, rye, sorghum, turf grass, sugarcane, and wheat.

8. The transgenic plant of claim 1, wherein said plant is a dicot plant is selected from the group consisting of alfalfa, Arabidopsis, barrel medic, banana, broccoli, bean, cabbage, canola, carrot, cassava, cauliflower, celery, citrus, cotton, a cucurbit, eucalyptus, garlic, grape, onion, lettuce, pea, peanut, pepper, potato, poplar, pine, sunflower, safflower, soybean, strawberry, sugar beet, sweet potato, tobacco, and tomato.

9. The transgenic plant of any one of claims 1-8, wherein said plant further comprises a second recombinant nucleic acid construct that provides for expression of MsDef1, MtDef2, MtDef4, Rs-AFP1, or Rs-AFP2.

10. The transgenic plant of any one of claims 1-9, wherein said heterologous signal peptide is a signal peptide of a plant gene.

11. The transgenic plant of claim 10, wherein said plant gene is a dicot or a monocot plant gene.

12. A transgenic plant cell obtained from the transgenic plant of any one of claims 1-11.

13. A transgenic plant seed obtained from the transgenic plant of any one of claims 1-11.

14. A processed food or feed composition obtained from either:
a) the transgenic plant seed of claim 13; or,
b) a transgenic plant part selected from the group consisting of a leaf, a stem, a flower, a root, and a tuber obtained from the transgenic plant of any one of claims 1-11.

15. The processed food or feed composition of claim 14, wherein said food or feed composition is a meal, a flour, an oil, or a starch.

16. The processed food or feed composition of claim 14, wherein mycotoxin levels in said food or feed composition are reduced by at least 50% relative to processed food or feed composition that lacks said recombinant nucleic acid construct.

17. The processed food or feed composition of claim 16, wherein mycotoxin levels in said food or feed composition are reduced by at least 75%, at least 85%, or at least 95% relative to processed food or feed composition that lacks said recombinant nucleic acid construct.

18. The processed food or feed composition of claim 16 or 17, wherein said mycotoxin is an aflatoxin, a fumonisin, a vomitoxin, or a trichothecene.

19. A method of producing the transgenic plant of any one of claims 1-11, comprising the steps of:
i) introducing a recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence into a plant, a plant cell, or a plant tissue; and
ii) selecting for a transgenic plant comprising said recombinant nucleic acid construct, wherein said transgenic plant selected in step (b) plant expresses said KP4 antifungal protein in the apoplast and provides for at least 50% inhibition of a plant pathogenic fungal infection relative to a control plant that lacks said recombinant nucleic acid construct.

20. The method of claim 19, wherein said transgenic plant is a monocot plant or a dicot plant.

21. The method of claim 19, wherein said nucleic acid construct is introduced into said plant, a plant cell or a plant tissue in step (a) by a method selected from the group consisting of particle bombardment, DNA transfection, DNA electroporation, Agrobacterium-mediated, Rhizobium-mediated, and Sinorhizobium-mediated transformation.

22. The method of claim 19, wherein said nucleic acid construct further comprises a sequence encoding a selectable marker and wherein said transgenic plant is obtained in step (b) by growing said plant, plant cell, or plant tissue under conditions requiring expression of said selectable marker for plant growth.

23. The method of claim 19, wherein said plant pathogenic fungus is selected from the group consisting of an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Magnaporthe grisea, Macrophomina sp., a Nectria sp., a Peronospora sp., Phakopsora pachyrhizi, Phialophora gregata, a Phoma sp., a Phymatotrichum sp., a Phytophthora sp., a Plasrnopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., Stenocarpella maydis, a Thielaviopsis sp., an Uncinula sp, a Venturia sp., and a Verticillium sp.

24. A method for obtaining the transgenic seed of claim 13, comprising the steps of:
a) crossing a transgenic plant comprising a recombinant nucleic acid construct, said recombinant nucleic acid construct comprising a promoter that is operably linked to a nucleic acid encoding a heterologous signal peptide that is operably linked to a non-native nucleic acid encoding a KP4 antifungal protein that is operably linked to a polyadenylation sequence, wherein said transgenic plant expresses said KP4 protein in the apoplast and provides for at least 50% inhibition of a plant pathogenic fungal infection relative to a control plant that lacks said recombinant nucleic acid construct to a plant that lacks said recombinant nucleic acid construct; and,
b) harvesting seed from a pollen recipient from said cross of step (i), thereby obtaining said transgenic seed.

25. The method of claim 24, wherein said method further comprises the steps of
iii) selecting for said transgenic seed from said harvested seed; and/or
iv) screening for said transgenic seed from said harvested seed.

26. A method of inhibiting a plant pathogenic fungal infection of a transgenic plant comprising the steps of:
i. exposing a transgenic plant of any one of claims 1-11 to a plant pathogenic fungus; and,
ii. obtaining a transgenic plant that exhibits at least 50% inhibition of a plant pathogenic fungal infection relative to a control plant that lacks said recombinant nucleic acid construct.

27. The method of claim 26, wherein said plant pathogenic fungus is selected from the group consisting of an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Magnaporthe grisea, Macrophomina sp., a Nectria sp., a Peronospora sp., Phakopsora pachyrhizi, Phialophora gregata, a Phoma sp., a Phymatotrichum sp., a Phytophthora sp., a Plasrnopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., Stenocarpella maydis, a Thielaviopsis sp., an Uncinula sp, a Venturia sp., and a Verticillium sp.

28. The method of claim 26 or 27, wherein said transgenic plant provides for at least 75%, at least 85%. or at least 95% inhibition of a plant pathogenic fungal infection relative to a control plant that lacks said recombinant nucleic acid construct.

29. The method of claim 26, wherein said method further comprises the step of harvesting at least one plant part selected from the group consisting of a leaf, a stem, a flower, a root, a tuber, or a seed from said plant obtained in step (ii).

30. The method of claim 29, wherein mycotoxin levels in said plant part are reduced by at least 50%, at least 75%, at least 85%, or at least 95% relative to a plant part obtained from a control plant that lacks said recombinant nucleic acid construct.

31. The method of claim 29, wherein said when said method further comprises the step of obtaining a processed food or feed composition from said plant part.

32. The method of claim 31, wherein mycotoxin levels in said processed food or feed composition from said plant part are reduced by at least 50%, at least 75%, at least 85%, or at least 95% relative to a processed food or feed composition obtained from a plant part of a control plant that lacks said recombinant nucleic acid construct.

33. A transgenic maize plant comprising a chromosome containing an insertion of a KP4 protein expression cassette of plasmid pZP212-KP4, wherein said insertion provides for at least 50% inhibition of a plant pathogenic fungal infection relative to a control maize plant that lacks said recombinant nucleic acid construct.

34. The transgenic maize plant of claim 33, wherein said transgenic maize plant is selected from the group consisting of transgenic maize plant lines 851, 947, 1040, 885, and 8510.

35. A transgenic soybean plant comprising a chromosome containing an insertion of an FMV promoter to a chimeric KP4 protein encoding region of SEQ ID NO:7, wherein said insertion provides for at least 50% inhibition of a plant pathogenic fungal infection relative to a control soybean plant that lacks said recombinant nucleic acid construct.

36. The transgenic soybean plant of claim 35, wherein said transgenic soybean plant is selected from the group consisting of transgenic soybean plant lines 2, 3, 7, 9, 10, 14, and 16.
